(19) United States
(12) Patent Application Publication
(10) Pub. No.: US 2021/0338671 A1
(43) Pub. Date: Nov. 4, 2021

(54) COMPOSITIONS OF SURFACE-MODIFIED THERAPEUTICALLY ACTIVE PARTICLES BY ULTRA-RAPID FREEZING

(71) Applicant: BOARD OF REGENTS, THE UNIVERSITY OF TEXAS SYSTEM, Austin, TX (US)

(72) Inventors: Robert O. WILLIAMS, III, Austin, TX (US); Chaeho MOON, Austin, TX (US); Alan B. WATTS, Austin, TX (US); John J. KOLENG, Austin, TX (US)

(21) Appl. No.: 17/262,313

(22) PCT Filed: Jul. 24, 2019
(86) PCT No.: PCT/US2019/043202

§ 371 Date: Jan. 22, 2021

Related U.S. Application Data
(60) Provisional application No. 62/702,674, filed on Jul. 24, 2018.

(51) Int. Cl. A61K 31/506; A61K 9/16; A61K 9/19; A61K 9/00

(52) U.S. Cl. CPC A61K 31/506; A61K 9/167; A61K 9/1623; A61K 9/1694; A61K 9/0075; A61K 9/19

(57) ABSTRACT

Pharmaceutical compositions which contain at less than 10% of an excipient and are presented as nanoaggregates are described herein. These pharmaceutical compositions have been shown to exhibit improved properties such as improved aerosolizability and aerodynamic performance. Also provided herein are methods of preparing the pharmaceutical compositions disclosed herein and use thereof.

This application claims the benefit of priority to U.S. Provisional Application No. 62/702,674, filed on Jul. 24, 2018, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a drug composition containing low amounts of excipients and therapeutic agents formulated as nanoaggregates.

### 2. Description of Related Art

Until recently, delivery of aerosolized antifungal drugs to the lungs was limited to amphotericin B (Le and Schiller, 2010; Borro et al. 2008). However, Hilberg et al. 2008 reported that inhaled voriconazole is more efficacious for treatment of invasive pulmonary aspergillosis (IPA) over that of inhaled amphotericin B, confirming that nebulized voriconazole formulation, initially reported by Tolman et al. 2009a, successfully treated patients with IPA who had previously failed with oral or injectable dosage forms of voriconazole with or without inhaled amphotericin B.

Tolman et al. reported inhaled voriconazole delivered to the lungs by nebulization (Tolman et al. 2009a; Tolman et al. 2009b). However, the concentration of voriconazole in lung tissue decreased after 6 hours to levels below the minimum detectable range (Tolman et al. 2009a). In addition, the potency of the nebulized formulation was also very low, only 5.9% (w/w) with sulfobutylether-β-cyclodextrin sodium (SBECD) as an excipient. The safety of SBECD delivered by pulmonary route has not been confirmed yet, and this high amount of inactive ingredient can cause serious side effects (Wong 1993). Voriconazole formulations for dry powder inhalation (DPI) were reported using poly-lactide-co-glycolide nanoparticles by Sinha et al. (Sinha et al. 2013) and poly-lactide microparticles by Arora et al. (Arora et al. 2015), but the drug loading was low for these particles (31% and 20% w/w, respectively). Arora et al. reported another voriconazole powder formulation for DPI using leucine as an excipient (Arora et al. 2016). However, all of these DPI powder formulations include non-GRAS excipients that have not been used for inhaled drugs approved by FDA. Beinborn et al. also developed amorphous and crystalline voriconazole formulations suitable for dry powder inhalation using the particle engineering technology, thin film freezing (TFF) (Beinborn et al. 2012a; Beinborn et al. 2012b). However, the amorphous formulation contained 75% (w/w) excipient and therefore has low potency, and the drug absorption efficiency was low with rapid clearance based on in vivo pharmacokinetic data in a mouse model. The AUC₀₋₂₄ₕ of the crystalline formulation was significantly higher than that of the amorphous formulation in both lung (452.6 μg·h/g and 232.1 μg·h/g, respectively) and plasma (38.4 μg·h/g and 18.6 μg·h/g, respectively). However, aerosol performance of the crystalline formulation was inferior (FPF 37.8%).

Recently, it has been proposed based upon modeling that nanoaggregates containing drug nanoparticles are more advantageously distributed with increased epithelial coverage in the lungs as compared to discrete micron-size particles and nanoparticles (Longest and Hindle 2017). An aggregate is a solid substance in particulate form made up of an assembly of particles held together by strong inter- or intramolecular cohesive forces (Chiou and Riegelman 1971). When three different forms of particulate drug were tested in the computational model, including conventional microparticles, nanoaggregates, and a true nanoaerosol of budesonide and fluticasone propionate, the total absorption efficiency of nanoaggregates of fluticasone propionate presented 57-fold higher than that of conventional microparticles. Although true nanoaerosol achieved better absorption efficiency, there are no practical devices available to deliver true nanoaerosols to the small airways therefore nanoaggregates provided the best approach to targeting drugs to the small airways. Slowly dissolving nanoaggregates were described as having improved drug uptake and distribution based on Longest et al. (Longest and Hindle 2017).

TFF is a particle engineering technology that employs an ultra rapid freezing rate of up to 10,000 K/sec (Engstrom et al. 2008). Due to the high degree of supercooling, TFF was successfully utilized to produce nanostructured aggregates (Sinswat et al. 2008). Spray drying is another common technique to produce micro- or nano-scale particles for DPI. However, particle formation during the drying process of spray drying generally takes longer (Wisniewski 2015) than the freezing process of TFF, allowing particles more time to grow, generating larger size of particles. Accordingly, typical spray drying methods will not have advantages of enhanced uptake and microdosimetry, which nanoaggregates have as described by Longest and Hindle 2017. Therefore, there remains a need to develop additional pharmaceutical compositions as a nanoaggregate which show improved properties such as enhanced aerosolization.

### SUMMARY OF THE INVENTION

The present disclosure provides pharmaceutical compositions comprising therapeutic agents and excipients as nanoaggregates, methods for their manufacture, and methods for their use. In some embodiments, the present disclosure provides pharmaceutical compositions comprising:
(A) a therapeutic agent; and
(B) an excipient, wherein the excipient comprises less than about 10% by weight of the pharmaceutical composition;
   wherein the pharmaceutical composition is formulated as a nanoaggregate comprising nanoparticles of the therapeutic agent and the surface of the nanoparticles of the therapeutic agent contains discrete domains of the excipient and wherein the discrete domains of the excipient reduce the contact area between the nanoparticles of the therapeutic agent.

In some embodiments, the therapeutic agent is present in a crystalline form. In other embodiments, the therapeutic agent is present in an amorphous form. In some embodiments, the excipient comprises from about 9% w/w to about 1% w/w of the pharmaceutical composition such as from about 6% w/w to about 2% w/w of the pharmaceutical composition. In some embodiments, the excipient comprises about 3% w/w of the pharmaceutical composition. In other embodiments, the excipient comprises about 5% w/w of the pharmaceutical composition.

In some embodiments, the discrete domains of the excipient comprise one or more non-continuous domains of the excipient on the surface. In other embodiments, the discrete domains of the excipient comprise a contiguous and continuous layer of the excipient. In some embodiments, the excipient is water-soluble. In some embodiments, the excipient is a solid at room temperature. In some embodiments, the excipient is a sugar alcohol such as mannitol. In some embodiments, the excipient is present as a nano-domain in the pharmaceutical composition. In some embodiments, the nano-domain of the excipient have a size from about 50 nm to about 500 nm such as from about 100 nm to about 200 nm.

In some embodiments, the pharmaceutical composition has a mass median aerodynamic diameter from about 1.5 to about 7.5 μm such as from about 2.5 to about 6.5 μm. In some embodiments, the pharmaceutical composition does not include a wax excipient. In some embodiments, the pharmaceutical composition does not include a hydrophobic excipient. In some embodiments, the therapeutic agent is selected from the group comprising anticancer agents, antifungal agents, psychiatric agents such as analgesics, consciousness level-altering agents such as anesthetic agents or hypnotics, nonsteroidal anti-inflammatory drugs (NSAIDS), anthelminthics, beta agonists, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives. In some embodiments, the therapeutic agent is an antifungal agent such as an azole antifungal drug. In some embodiments, the azole antifungal drug is voriconazole. In some embodiments, the pharmaceutical composition further comprises one or more additional excipients. In some embodiments, the pharmaceutical composition further comprises one or more additional therapeutic agents.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transdermally, vaginally, in crèmes, in lipid compositions, via a catheter, via a lavage, via continuous infusion, via infusion, via inhalation, via injection, via local delivery, or via localized perfusion. In some embodiments, pharmaceutical composition is formulated for administration via inhalation.

In some embodiments, the pharmaceutical composition is formulated for use with an inhaler such as a fixed dose combination inhaler, a single dose dry powder inhaler, a multi-dose dry powder inhaler, multi-unit dose dry powder inhaler, a metered dose inhaler, or a pressurized metered dose inhaler. In some embodiments, the inhaler is a capsule-based inhaler. In some embodiments, the inhaler is a low resistance inhaler. In other embodiments, the inhaler is a high resistance inhaler. In some embodiments, the inhaler is used with a flow rate from about 10 L/min to about 150 L/min such as from about 20 L/min to about 100 L/min. In some embodiments, the inhaler has a pressure differential is from 0.5 kPa to about 5 kPa. In some embodiments, the pressure differential is 1 kPa, 2 kPa, or 4 kPa. In some embodiments, the inhaler has a loaded dose from about 0.1 mg to about 50 mg. In some embodiments, the inhaler has a loaded dose from about 0.1 mg to about 10 mg. In other embodiments, the inhaler has a loaded dose from about 5 mg to about 50 mg such as from about 5 mg to about 25 mg. In some embodiments, the inhaler is configured to deliver one or a series of doses from one or more unit doses loaded sequentially. In some embodiments, the inhaler is configured to deliver one dose from one unit dose. In other embodiments, the inhaler is configured to deliver a series of doses from one unit dose. In other embodiments, the inhaler is configured to deliver one dose each from a series of capsules loaded sequentially. In other embodiments, the inhaler is configured to deliver a series of doses from a series of capsules loaded sequentially.

In still another aspect, the present disclosure provides methods of treating or preventing a disease or disorder in a patient in need thereof comprising administering to the patient a therapeutically effective amount of a pharmaceutical composition described herein comprising a therapeutic agent effective to treat the disease or disorder. In some embodiments, the disease or disorder is in the lungs. In some embodiments, the disease or disorder is an infection such as an infection of a fungus. In some embodiments, the therapeutic agent is an anti-fungal agent such as an azole anti-fungal agent. In some embodiments, the therapeutic agent is voriconazole.

In still yet another aspect, the present disclosure provides methods of preparing a pharmaceutical composition comprising:
(A) admixing a therapeutic agent and an excipient wherein the excipient is present in an amount of less than 10% w/w with a solvent to form a precursor solution;
(B) depositing the precursor solution onto a surface at a temperature suitable to cause the solvent to freeze; and
(C) removing the solvent to obtain a pharmaceutical composition.

In some embodiments, the solvent is a mixture of two or more solvents. In some embodiments, the mixture of solvents comprises water. In some embodiments, the solvent is an organic solvent. In some embodiments, the organic solvent is acetonitrile. In other embodiments, the organic solvent is 1,4-dioxane. In some embodiments, the solvent is a mixture of water and an organic solvent such as a mixture of water and acetonitrile. In some embodiments, the mixture of two or more solvents comprises from about 10% v/v to about 90% v/v of the organic solvent. In some embodiments, the mixture comprises from about 40% v/v to about 60% v/v of the organic solvent such as about 50% v/v of the organic solvent. In other embodiments, the mixture comprises from about 20% v/v to about 40% v/v of the organic solvent such as about 30% v/v of the organic solvent. In some embodiments, the therapeutic agent and excipient comprises less than 10% w/v of the precursor solution such as from about 0.5% to about 5% w/v of the precursor solution. In some embodiments, the therapeutic agent and excipient comprises about 1% w/v of the precursor solution. In other embodiments, the therapeutic agent and excipient comprises about 3% w/v of the precursor solution.

In some embodiments, the surface is rotating. In some embodiments, the temperature is from about 0° C. to about −200° C. In some embodiments, the temperature is from about 0° C. to about −120° C. such as from about −50° C. to about −90° C. In some embodiments, the temperature is about −60° C. In other embodiments, the temperature is from about −125° C. to about −175° C. such as about −150° C. In some embodiments, the solvent is removed at reduced pressure. In some embodiments, the solvent is removed via lyophilization. In some embodiments, the lyophilization is carried out at a lyophilization temperature from about −20° C. to about −100° C. such as about −40° C. In some embodiments, the reduced pressure is less than 250 mTorr such as about 100 mTorr.

In some embodiments, the methods further comprise heating the pharmaceutical composition at reduced pressure. In some embodiments, the pharmaceutical composition is heated to a temperature from about 0° C. to about 30° C. such as about room temperature or about 25° C. In some embodiments, the reduced pressure is less than 250 mTorr such as about 100 mTorr. In some embodiments, the reduced pressure is the same as the reduced pressure during the lyophilization.

In still yet another aspect, the present disclosure provides pharmaceutical compositions prepared according to the methods described herein.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows XRPD of (a) Voriconazole powder; (b) TFF-VCZ; (c) TFF-VCZ-MAN 95:5; (d) TFF-VCZ-MAN 70:30; (e) TFF-VCZ-MAN 50:50; (f) TFF-MAN.

FIG. 2 shows modulated DSC of (a) TFF-MAN; (b) TFF-VCZ; (c) TFF-VCZ-MAN 95:5; (d) TFF-VCZ-MAN 50:50.

FIGS. 3A-3J show SEM images of TFF-VCZ-MAN: (FIG. 3A) TFF-VCZ; (FIG. 3B) TFF-VCZ-MAN 95:5; (FIG. 3C) TFF-VCZ-MAN 70:30; (FIG. 3D) TFF-VCZ-MAN 50:50; (FIG. 3E) TFF-VCZ-MAN 25:75; (FIG. 3F) TFF-MAN; (FIG. 3G) aerosolized TFF-VCZ-MAN 95:5; (FIG. 3H) aerosolized TFF-VCZ-MAN 50:50; (FIG. 3I) TFF-VCZ-MAN 25:75, after 5 min in Franz cells; (FIG. 3J) TFF-VCZ-MAN 95:5, after 5 min in Franz cells.

FIGS. 4A-4F show SEM images of: (FIG. 4A) TFF-VCZ; (FIG. 4B) TFF-VCZ-MAN 95:5, 3D topography image of: (FIG. 4C) TFF-VCZ; (FIG. 4D) TFF-VCZ-MAN 95:5, and illustration of contact area and distance between particles of: (FIG. 4E) TFF-VCZ; (FIG. 4F) TFF-VCZ-MAN 95:5.

FIG. 5 shows AFM topography image of aerosolized TFF-VCZ-MAN 95:5 by DP4 insufflator.

FIG. 6 shows SSA of TFF-VCZ-MAN powder formulations (n=3; mean±SD).

FIGS. 7A-7C show SEM/EDX data of TFF-VCZ-MAN 50:50: (FIG. 7A) SEM image; (FIG. 7B) elemental analysis of spot A; (FIG. 7C) elemental analysis of spot B.

FIGS. 8A & 8B show FT-IR (FIG. 8A, 3500 cm⁻¹ to 3100 cm⁻¹ region; FIG. 8B, 1290 cm⁻¹ to 1230 cm⁻¹ region) of (a) voriconazole Powder; (b) TFF-VCZ; (c) TFF-VCZ-MAN 95:5; (d) TFF-VCZ-MAN 70:30; (e) TFF-VCZ-MAN 50:50; (f) TFF-MAN.

FIGS. 9A & 9B show 1D CP-MAS spectrum of (FIG. 9A) TFF-VCZ; and

(FIG. 9B) TFF-VCZ-MAN 90:10; ¹³C spectrum (left spectrum) and ¹⁹F spectrum (right spectrum).

FIGS. 10A & 10B show 2D ¹H-¹³C HETCOR spectra of (FIG. 10A) TFF-VCZ; and (FIG. 10B) TFF-VCZ-MAN 90:10.

FIG. 11 shows FPF (% of metered) of TFF-VCZ-MAN dry powder formulations (n=3; mean±SD).

FIG. 12 shows aerodynamic particle size distribution profile of TFF-VCZ-MAN 95:5 by time sheared: at 0 min; at 15 min; at 30 min; at 60 min from left to right (n=3; mean±SD).

FIGS. 13A & 13B show aerodynamic properties of TFF-VCZ-MAN 95:5 by time sheared: (line a) FPF, % of delivered; (line b) FPF, % of metered; (line c) MMAD; and (line d) GSD (n=3; mean±SD).

FIGS. 14A & 14B show aerodynamic properties of TFF-VCZ-MAN 95:5 by time stored at 25° C./60% RH: (line a) FPF, % of delivered; (line b) FPF, % of metered; (line c) MMAD; and (line d) GSD (n=3; mean±SD).

FIG. 15 shows cumulative voriconazole release (%) of (line a) TFF-VCZ-PVPK25 25:75 (amorphous); (line b) TFF-VCZ-MAN 25:75; (line c) TFF-VCZ-MAN 50:50; (line d) TFF-VCZ-MAN 95:5 (n=3; mean±SD).

FIG. 16 shows images of the freezing process.

FIGS. 17A-17D show AFM topography image of: (a) formulation #2 (scale 5 μm×5 μm), and (b) formulation #4 (scale 2 μm×2 μm); and corresponding 3D topography image of: (c) formulation #2, and (d) formulation #4.

FIGS. 18A-18F show SEM images of voriconazole nanoaggregates: (a) formulation #1, (b) formulation #2, (c) formulation #3, (d) formulation #4, (e) formulation #5, and (f) formulation #6.

FIGS. 19A-19F show SEM images of aerosolized voriconazole nanoaggregates: (a)-(b) formulation #7 and (c)-(f) formulation #6.

FIG. 20 shows XRPD of (a) voriconazole powder, (b) TFF-voriconazole, (c) formulation #6 (small scale), (d) formulation #6 (large scale), and (e) TFF-mannitol.

### DESCRIPTION OF ILLUSTRATIVE EMBODIMENTS

In some aspects of the present disclosure, the pharmaceutical compositions contain nanoaggregates. These compositions may be prepared through methods such as thin-film freezing and contain a therapeutic agent and an excipient. In some embodiments, these composition also show improved aerosolization or other pharmaceutical properties are provided.

Also provided herein are methods of preparing and using these compositions. Details of these compositions are provided in more detail below.

### I. PHARMACEUTICAL COMPOSITIONS

In some aspects, the present disclosure provides pharmaceutical compositions containing a therapeutic agent and an excipient, wherein the excipient comprises less than about 10% w/w of the composition. These pharmaceutical compositions may further comprise one or more additional therapeutic agents or one or more additional excipients. Such compositions may be prepared using such methods as thin film freezing. These methods include freezing a solution of the therapeutic agent and the excipient in a solvent and then removing that solvent either in reduced pressure and/or reduced temperature. Methods of preparing pharmaceutical compositions using thin film freezing are described in U.S. Patent Application No. 2010/0221343, Watts, et al., 2013, Engstrom et al. 2008, Wang et al. 2014, Thakkar at el. 2017, O'Donnell et al. 2013, Lang et al. 2014a, Lang et al. 2014b, Carvalho et al. 2014, Beinborn et al. 2012a, Beinborn et al. 2012b, Zhang et al. 2012, Overhoff et al. 2008, Overhoff et al. 2007a, Overhoff et al. 2007b, Watts et al. 2010, Yang et al. 2010, DiNunzio et al. 2008, Yang et al. 2008, Purvis et al. 2007, Liu et al. 2015, Sinswat et al. 2008, and U.S. Pat. No. 8,968,786, all of which are incorporated herein by reference.

Such pharmaceutical compositions may be present as a nanoaggregate which comprises an assembly of nano-particles which are attracted or joined together through inter or intramolecular cohesive forces. In the pharmaceutical compositions described herein, the nanoaggregates may comprise one or more particles of the drug which is coated with a discrete non-continuous nano-domains of the excipient. Without wishing to be bound by any theory, it is believed that the nano-domains of the excipient may comprise a size from about 25 nm, 50 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, 475 nm, 500 nm, 525 nm, 550 nm, 575 nm, 600 nm, 625 nm, or 650 nm, or any range derivable therein. The size of these nano-domains of the excipient comprise a size from about 25 nm to about 750 nm, from about 50 nm to about 500 nm, or from about 100 nm to about 200 nm. Without wishing to be bound by any theory, it is believed that these nano-domains may be present as discrete compositions dotting the surface of a nanoaggregate that comprises of the therapeutic agent. The pharmaceutical compositions may further comprise a mass median aerodynamic diameter from about 2.5 μm to about 7.5 μm, from about 3.0 μm to about 6.0 μm, from about 4.0 μm to about 6.0 μm, or from about 2.5, 2.75, 3.0, 3.25, 3.5, 3.75, 4, 4.25, 4.5, 4.75, 5, 5.25, 5.5, 5.75, 6.0, 6.25, 6.5, 6.75, 7.0, 7.25, to about 7.5 μm, or any range derivable therein.

### A. Therapeutic Agent

The “therapeutic agent” used in the present methods and compositions refers to any substance, compound, drug, medicament, or other primary active ingredient that provides a therapeutic or pharmacological effect when administered to a human or animal. When a therapeutic agent is present in the composition, the therapeutic agent is present in the composition at a level between about 50% to about 99% w/w, between about 70% to about 99% w/w, between about 90% to about 97% w/w, or between about 95% to about 97% w/w of the total composition. In some embodiments, the amount of the therapeutic agent is from about 50%, 60%, 70%, 80%, 90%, 91%, 92%, 93%, 94%, 95%, 96%, 97%, to about 99% w/w or any range derivable therein.

Suitable lipophilic therapeutic agents may be any poorly water-soluble, biologically active agents or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal antiinflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta agonists, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the therapeutic agents may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the therapeutic agents may be voriconazole or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, posaconazole, voriconazole, terconazole and c) thiazoles such as abafungin. Other drugs that may be used with this approach include, but are not limited to, hyperthyroid drugs such as carimazole, anticancer agents like cytotoxic agents such as epipodophyllotoxin derivatives, taxanes, bleomycin, anthracyclines, as well as platinum compounds and camptothecin analogs. The following therapeutic agents may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The agents may also include a psychiatric agent such as an antipsychotic, anti-depressive agent, or analgesic and/or tranquilizing agents such as benzodiazepines. The agents may also include a consciousness level-altering agent or an anesthetic agent, such as propofol. The present compositions and the methods of making them may be used to prepare a pharmaceutical compositions with the appropriate pharmacokinetic properties for use as therapeutics.

In some embodiments, the compositions described herein may include a long acting β agonist (LABA). Some non-limiting examples of long acting β-agonist include formoterol such as formoterol fumarate, salmeterol such as salmeterol xinafoate, bambuterol, clenbuterol, indacaterol, olodaterol, protokylol, abediterol, salmefamol, vilanterol, arformoterol, carmoterol, PF-610355, GSK-159797, GSK-597901, GSK-159802, GSK-642444, GSK-678007, or other long acting β-agonist known in the art.

In other embodiments, the composition described herein may include a long acting muscarinic antagonist (LAMA). Some non-limiting examples of long acting muscarinic antagonist include salts of tiotropium, aclidinium, dexpirronium, ipratropium, oxitropium, darotropium, glycopyrronium, or glycopyrrolate derivative or other long acting muscarinic antagonist known in the art such as those taught by US Patent Application No. 2009/0181935, PCT Patent Application No. WO 2010/007561, and PCT Patent Application No. WO 2008/035157, which are incorporated herein by reference.

In other embodiments, the compositions described herein may include a corticosteroid, specifically a corticosteroid suitable for inhalation. Some non-limiting examples of corticosteroid include beclomethasone dipropionate, budesonide, flunisolide, fluticasone propionate, fluticasone furoate, mometasone furoate, ciclesonide, rofleponide palmitate, triamcinolone acetonide, or other corticosteroid known in the art.

In other embodiments, the composition described herein may comprise one or more antibiotic agents. Some classes of antibiotics include penicillins, cephalosporins, carbapenems, macrolides, aminoglycosides, quinolones (including fluoroquinolones), sulfonamides and tetracylcines. In some embodiments, the compositions may comprise a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compositions comprise a drug which acts against mycobacteria such as cycloserine, capreomycin, ethionamide, rifampicin, rifabutin, rifapentine, and streptomycin. Other antibiotics that are contemplated may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

In some embodiments, the compositions may further comprise one or more anti-fungal agents such as those described above. Some anti-fungal agents include, but are not limited to, amphotericin B, an azole anti-fungal compound, echinocandins, or flucytosine. Some non-limiting examples of azole anti-fungal compounds include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, posaconazole, voriconazole, terconazole and c) thiazoles such as abafungin.

In some embodiments, the composition may further comprise one or more anti-viral agents such as nucleoside analogs such as acyclovir, famciclovir, valaciclovir, penciclovir, and ganciclovir or other antiviral agents such as a pegylated interferon, interferon alfa-2b, lamivudine, adefovir, telbivudine, entercavir, or tenofovir

### B. Excipients

In some aspects, the present disclosure comprises one or more excipients formulated into pharmaceutical compositions. In some embodiments, the excipients used herein are water soluble excipients. These water soluble excipients include saccharides such as disaccharides such as sucrose, trehalose, or lactose, a trisaccharide such as fructose, glucose, galactose, or raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In some aspects, the present pharmaceutical compositions may further exclude a hydrophobic or waxy excipient such as waxes and oils. Some non-limiting examples of hydrophobic excipients include hydrogenated oils and partially hydrogenated oils, palm oil, soybean oil, castor oil, carnauba wax, beeswax, palm wax, white wax, castor wax, or lanoline. Additionally, the present disclosure may further comprise one or more amino acids or an amide or ester derivative thereof. In some embodiments, the amino acids used may be one of the 20 canonical amino acids such as glycine, alanine, valine, isoleucine, leucine, methionine, phenylalanine, tyrosine, tryptophan, serine, threonine, asparagine, glutamine, cysteine, selenocysteine, proline, arginine, histidine, lysine, aspartic acid, or glutamic acid. These amino acids may be in the D or L orientation or the amino acids may be an α-, β-, γ-, or δ-amino acids. In other embodiments, one of the common non-canonical amino acids may be used such as carnitine, GABA, carboxyglutamic acid, levothyroxine, hydroxyproline, seleonmethionine, beta alanine, ornithine, citrulline, dehydroalanine, δ-aminolevulinic acid, or 2-aminoisobutyric acid.

In some aspects, the amount of the excipient in the pharmaceutical composition is from about 0.5% to about 10% w/w, from about 1% to about 10% w/w, from about 2% to about 8% w/w, or from about 2% to about 5% w/w. The amount of the excipient in the pharmaceutical composition comprises from about 0.5%, 0.75%, 1%, 1.25%, 1.5%, 1.75%, 2%, 2.5%, 3%, 3.5%, 4%, 4.5%, 5%, 6%, 7%, 8%, 9%, to about 10% w/w, or any range derivable therein, of the total pharmaceutical composition. In one embodiment, the amount of the excipient in the pharmaceutical composition is at 2% to 5% w/w of the total weight of the pharmaceutical composition.

### II. MANUFACTURING METHODS

Thus, in one aspect, the present disclosure provides pharmaceutical compositions which may be prepared using a thin-film freezing process. Such methods are described in U.S. Patent Application No. 2010/0221343 and Watts, et al., 2013, both of which are incorporated herein by reference. In some embodiments, these methods involve dissolving the components of the pharmaceutical composition into a solvent to form a precursor solution. The solvents may be either water or an organic solvent. Some non-limiting examples of organic solvents which may be used include volatile organic solvent such as 1,4-dioxane, acetonitrile, acetone, methanol, ethanol, isopropanol, dichloromethane, chloroform, tetrahydrofuran, tert-butyl alcohol, dimethyl sulfoxide, N,N-dimethyl formamide, diethyl ether, ethyl acetate, isopropyl acetate, butyl acetate, propyl acetate, toluene, hexanes, heptane, pentane, or combinations thereof. In some embodiments, the precursor solution may contain less than 10% w/v of the therapeutic agent and excipient. The precursor solution may contain less than 0.5%, 1%, 2%, 3%, 4%, 5%, 6%, 7%, 8%, 9%, or 10% w/v, or any range derivable therein.

This precursor solution may be deposited on a surface which is at a temperature that causes the precursor solution to freeze. In some embodiments, this temperature may be below the freezing point of the solution at ambient pressure. In other embodiments, a reduced pressure may be applied to the surface causing the solution to freeze at a temperature below the ambient pressure's freezing point. The surface may also be rotating or moving on a moving conveyer-type system thus allowing the precursor solution to distribute evenly on the surface. Alternatively, the precursor solution may be applied to surface in such a manner to generate an even surface.

After the precursor solution has been applied to the surface, the solvent may be removed to obtain a pharmaceutical composition. Any appropriate method of removing the solvent may be applied including evaporation under reduced pressure or elevated temperature or lyophilization. In some embodiments, the lyophilization may comprise a reduced pressure and/or a reduced temperature. Such a reduced temperature may be from 25° C. to about −200° C., from 20° C. to about −175° C., from about 20° C. to about 150° C., from 0° C. to about −125° C., from −20° C. to about −100° C., from −75° C. to about 175° C., or from ˜100° C. to about −160° C. The temperature is from about −20° C., −30° C., 35° C., −40° C., −45° C., −50° C., −55° C., −60° C., −70° C., −80° C., −90° C., −100° C., −110° C., −120° C., −130° C., −140° C., −150° C., −160° C., −170° C., −180° C., −190° C., to about 200° C., or any range derivable therein. Additionally, the solvent may be removed at a reduced pressure of less than 500 mTorr, 450 mTorr, 400 mTorr, 375 mTorr, 350 mTorr, 325 mTorr, 300 mTorr, 275 mTorr, 250 mTorr, 225 mTorr, 200 mTorr, 175 mTorr, 150 mTorr, 125 mTorr, 100 mTorr, 75 mTorr, 50 mTorr, or 25 mTorr.

Such as composition prepared using these methods may exhibit a brittle nature such that the composition is easily sheared into smaller particles when processed through a device. These compositions have high surface areas as well as exhibit improved flowability of the composition. Such flowability may be measured, for example, by the Carr index or other similar measurements. In particular, the Can's index may be measured by comparing the bulk density of the powder with the tapped density of the powder. Such compounds may exhibit a favorable Carr index and may result in the particles being better sheared to give smaller particles when the composition is processed through a secondary device to deliver the drug.

### III. DEFINITIONS

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

As used herein, the term “domain” refers to a specific area of the composition comprise substantially of a single material distinct in characteristics from the other components of the composition. A “discrete domain” refers to an individual area of the composition which is different and separate from each other area of the composition. The domain may substantially consist of a single element from the composition. These domains may be non-continuous such that the discrete domains are present as multiple domains which do not touch each other.

As used herein, the term “nanoparticle” has its customary and ordinary definition and refers to discrete particles which behave as a whole unit rather than as individual molecules within the particle. A nanoparticle may have a size from about 1 to about 10,000 nm with ultrafine nanoparticles having a size from 1 nm to 100 nm, fine particles having a size from 100 nm to 2,500 nm, and coarse particles having a size from 2,500 nm to 10,000 nm. In some embodiments, the nanoaggregates described herein may comprise a composition of multiple nanoparticles and have a size from about 10 nm to about 100 μm.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Discussion and Results

### A. Physicochemical Properties of Voriconazole Dry Powder Formulations

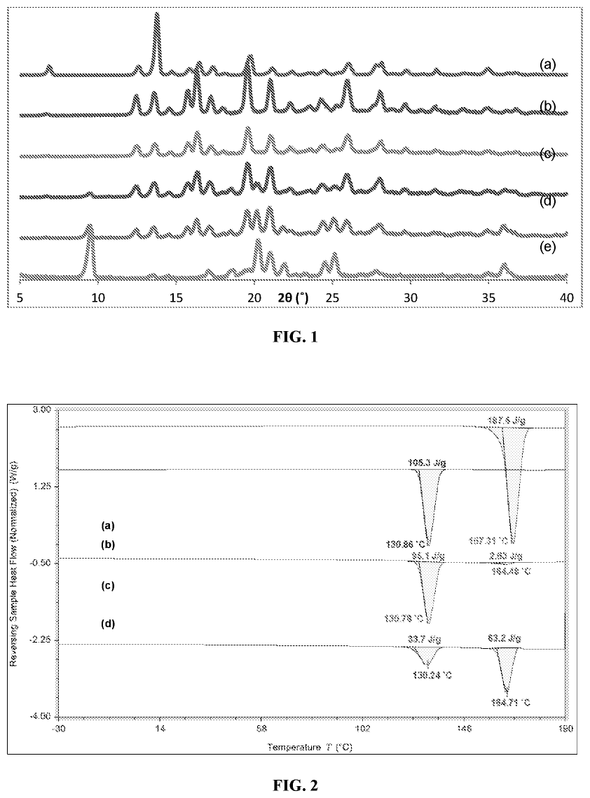

The TFF technology was used to produce crystalline voriconazole powder formulations containing mannitol (see Table 1). XRPD and mDSC were mainly employed to determine crystallinity of the formulations. TFF-VCZ powder formulations including mannitol were identified as crystalline as shown in FIGS. 1 and 2. The TFF-VCZ-MAN powder formulations exhibited characteristic voriconazole peaks of XRPD corresponding to voriconazole bulk powder (e.g., 12.4°2θ and 13.6°2θ) and δ-mannitol (e.g., 9.5 °2θ and 20.2°2θ) as shown in FIG. 1. These indicate that the powder formulations consist of crystalline voriconazole and δ-mannitol. The intensity of δ-mannitol peaks decreased as amounts of mannitol (% w/w) were reduced in the TFF-VCZ-MAN powder formulations, and the peaks corresponding to δ-mannitol were not detectable when the powder formulations contained 5% (w/w) mannitol. TFF-MAN dry powder was mainly δ-form, while trace amounts of α- and β-forms were detected by XRPD (13.5°2θ and 14.5°2θ respectively).

TABLE 1
Summary of voriconazole dry powder formulations investigated
using thin-film freezing (TFF) technology.

| | Drug:Excipient | Dissolved | Solvent |
|---|---|---|---|
| Sample | ratio (w/w) | solids | compositions |
| TFF-VCZ | No excipient | 1.0% (w/v) | Water:acetonitrile |
| | | | 50:50 (v/v) |
| TFF-VCZ-MAN | 99:1 | 1.0% (w/v) | Water:acetonitrile |
| 99:1 | | | 50:50 (v/v) |
| TFF-VCZ-MAN | 98:2 | 1.0% (w/v) | Water:acetonitrile |
| 98:2 | | | 50:50 (v/v) |
| TFF-VCZ-MAN | 97:3 | 1.0% (w/v) | Water:acetonitrile |
| 97:3 | | | 50:50 (v/v) |
| TFF-VCZ-MAN | 95:5 | 1.0% (w/v) | Water:acetonitrile |
| 95:5 | | | 50:50 (v/v) |
| TFF-VCZ-MAN | 93:7 | 1.0% (w/v) | Water:acetonitrile |
| 93:7 | | | 50:50 (v/v) |
| TFF-VCZ-MAN | 90:10 | 1.0% (w/v) | Water:acetonitrile |
| 90:10 | | | 50:50 (v/v) |
| TFF-VCZ-MAN | 85:15 | 1.0% (w/v) | Water:acetonitrile |
| 85:15 | | | 50:50 (v/v) |
| TFF-VCZ-MAN | 80:20 | 1.0% (w/v) | Water:acetonitrile |
| 80:20 | | | 50:50 (v/v) |
| TFF-VCZ-MAN | 70:30 | 1.0% (w/v) | Water:acetonitrile |
| 70:30 | | | 50:50 (v/v) |
| TFF-VCZ-MAN | 50:50 | 1.0% (w/v) | Water:acetonitrile |
| 50:50 | | | 50:50 (v/v) |
| TFF-VCZ-MAN | 25:75 | 1.0% (w/v) | Water:acetonitrile |
| 25:75 | | | 50:50 (v/v) |
| TFF-MAN | No drug | 1.0% (w/v) | Water:acetonitrile |
| | | | 50:50 (v/v) |
| TFF-VCZ-PVPK | 25:75 | 1.0% (w/v) | 1,4-dioxane |
| 25 | | | |

mDSC also confirmed crystallinity of the TFF-VCZ-MAN powder formulations. FIG. 2 shows no glass transition detected in the TFF-VCZ-MAN 95:5 and TFF-VCZ-MAN 50:50, but only endotherm peaks corresponding to melting of voriconazole and mannitol. TFF-VCZ had a melting endotherm peak at 130.86° C. with a heat of fusion of 105.3 J/g. When expected heats of fusions for voriconazole in TFF-VCZ-MAN powders are calculated by % fraction (w/w), the heats of fusions for voriconazole in TFF-VCZ-MAN 95:5 and TFF-VCZ-MAN 50:50 were 100.0 J/g and 52.6 J/g respectively. The measured heats of fusion for voriconazole were 95.1 J/g for TFF-VCZ-MAN 95:5, and 33.7 J/g for TFF-VCZ-MAN 50:50, and these were 95.1% and 64.0% of the expected values. TFF-MAN had a melting endotherm peak at 167.31° C. with a heat of fusion of 187.5 J/g. The expected heats of fusions for mannitol in TFF-VCZ-MAN 95:5 and TFF-VCZ-MAN 50:50 were 9.38 J/g and 93.8 J/g, respectively. The measured heats of fusion for mannitol were 2.63 J/g and 63.2 J/g, respectively, and these were 28.0% and 67.4% of the expected values. Table 2 presents composition ratios of voriconazole to mannitol (voriconazole:mannitol w/w) in the two formulations tested by mDSC. The ratios were calculated by integration of proton peaks using ¹H-NMR. Theoretical ratio of one proton for TFF-VCZ-MAN 95:5 is 1:0.1009, and the experimental ratio was calculated as 1:0.0992 that represented 98.3% of expected mannitol was found in TFF-VCZ-MAN 95:5. In the case of TFF-VCZ-MAN 50:50, 100% of expected mannitol was detected by ¹H-NMR.

TABLE 2
Quantitative comparison of voriconazole and mannitol
in TFF-VCZ-MAN 95:5 and TFF-VCZ-MAN 50:50 by ¹H-NMR.

| | | | | | | | Theoretical | Experimental |
|---|---|---|---|---|---|---|---|---|
| | | | | | | | 1H | 1H |
| | Voriconazole | | | Mannitol | | | integration | integration |
| | Chemical | Number | | Chemical | Number | | ratio | ratio |
| | shift (ppm) | of proton | Integration | shift (ppm) | of proton | Integration | (VCZ:MAN) | (VCZ:MAN) |
| TFF-VCZ- | 3.91 | 1 | 10.13 | 4.10 | 2 | 2.01 | 1:0.1009 | 1:0.0992 |
| MAN | | | | | | | | |
| (95:5) | | | | | | | | |
| TFF-VCZ- | 3.91 | 1 | 1.02 | 4.10 | 2 | 3.91 | 1:1.917 | 1:1.917 |
| MAN | | | | | | | | |
| (50:50) | | | | | | | | |

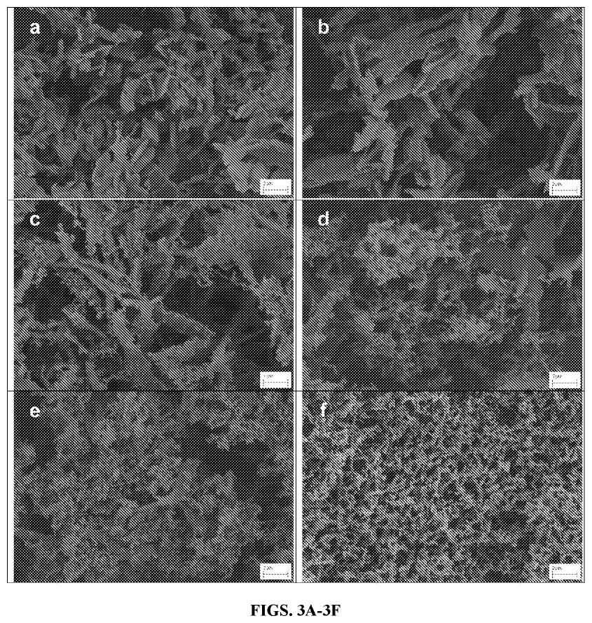

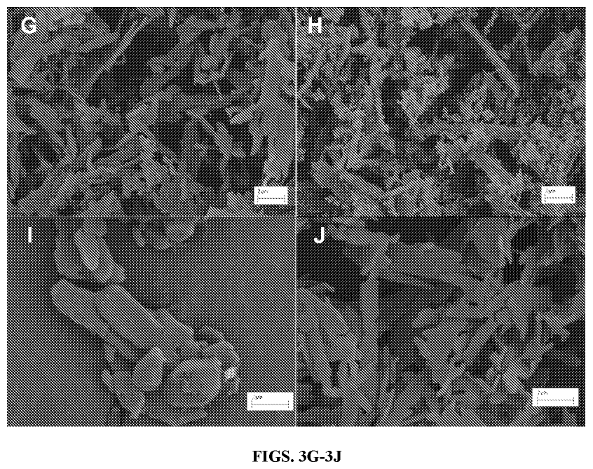

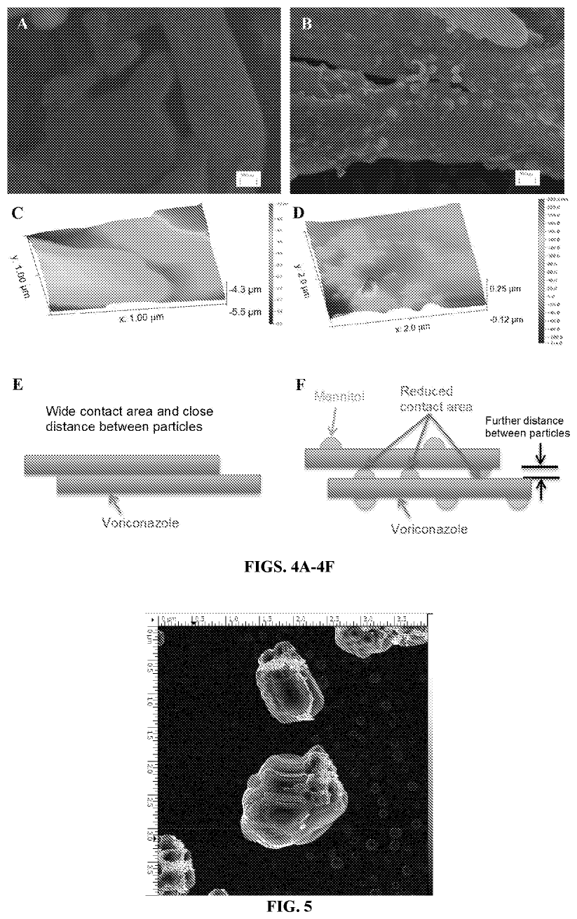

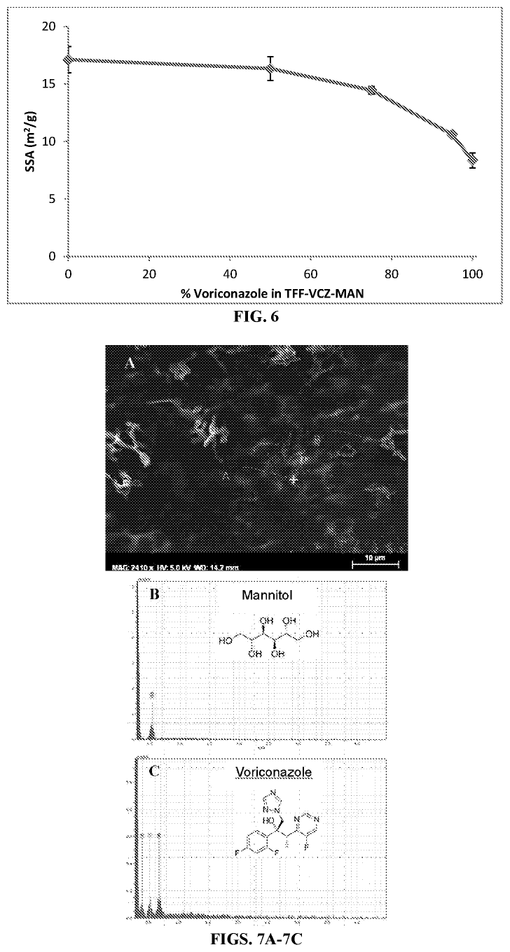

Particle morphology of TFF-VCZ-MAN powders is presented in FIG. 3. Agglomeration of micron-size particles was observed in the TFF-VCZ powders, and those particles were also found in other TFF-VCZ-MAN powder formulations. More porous matrix was observed with TFF-VCZ-MAN powders containing higher amounts of mannitol. 3D topography and illustration of TFF-VCZ and TFF-VCZ-MAN 95:5 powders shown in FIG. 4 confirms that the surface texture of TFF-VCZ-MAN 95:5 powders is rough, while that of TFF-VCZ powders is smooth. High resolution topography of TFF-VCZ-MAN 95:5 powders in FIG. 5 indicates that TFF-VCZ-MAN 95:5 powders are nanoaggregates consisting of about 150-500 nm nano-particles. SSAs of these TFF-VCZ-MAN powders are shown in FIG. 6. The TFF-VCZ powders indicated the lowest SSA (8.36 m²/g), and the porous matrix of TFF-MAN dry powder exhibited the highest SSA (17.11 m²/g). The SSA increased as more mannitol was added to TFF-VCZ-MAN powder formulations. By SEM/EDX shown in FIG. 7, the micron-size particles were identified as being composed of voriconazole nanoaggregates with detection of nitrogen, oxygen, and fluorine. The porous matrix was identified as mannitol by detection of oxygen without nitrogen and fluorine.

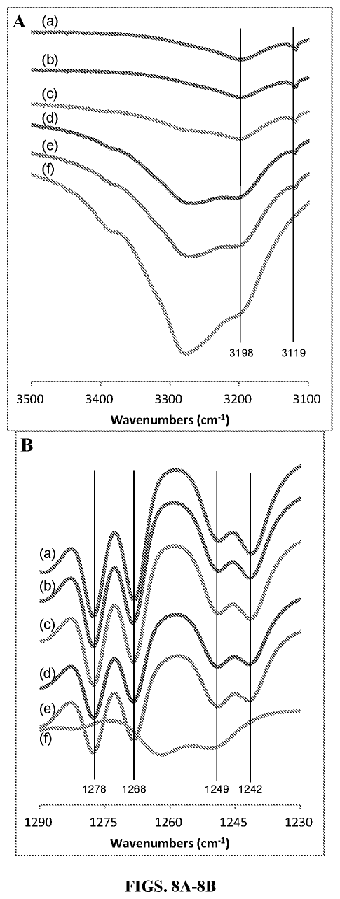

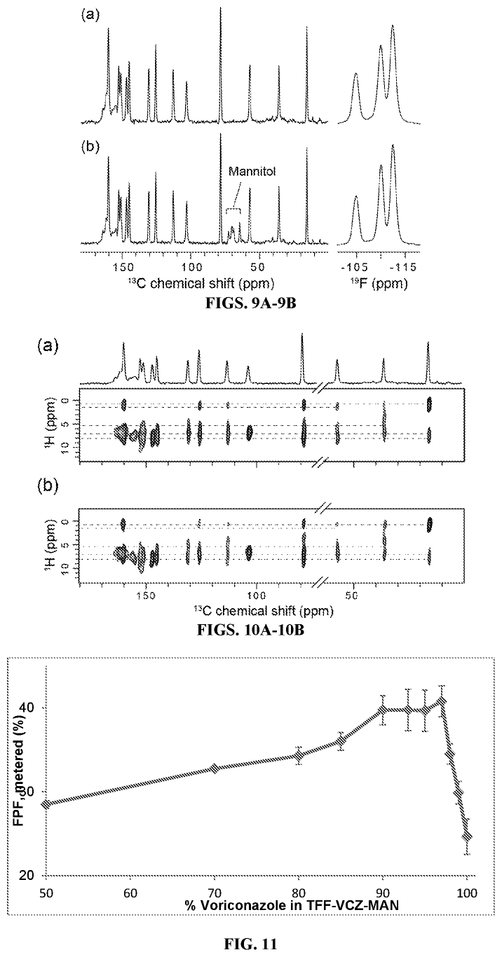

The FT-IR peak pattern of TFF-VCZ powder was matched with that of voriconazole bulk powder, and the same peak pattern was also found with TFF-VCZ-MAN powders containing different amounts of mannitol. The peak pattern of TFF-MAN was also found from TFF-VCZ-MAN powders. Therefore, the peaks only corresponding to TFF-VCZ and TFF-MAN were observed in TFF-VCZ-MAN powders, and no new peak was found on the FT-IR spectrum of TFF-VCZ-MAN powders, as shown in FIG. 8. 1D ¹³C and ¹⁹F CP-MAS spectra by ssNMR are shown in FIG. 9. Voriconazole has no spectral overlap of ¹³C peaks with mannitol and possesses all resonances in the ¹⁹F spectra. It shows identical spectra in TFF-VCZ and TFF-VCZ-MAN. Moreover, the sharp ¹³C and ¹⁹F peaks in the spectra of TFF-VCZ-MAN 90:10 confirm the crystallinity of both voriconazole and mannitol. 2D ¹H-¹³C HETCOR spectrum of TFF-VCZ-MAN 90:10 was compared with spectrum of TFF-VCZ in FIG. 10. Intermolecular cross-peaks between voriconazole and mannitol from TFF-VCZ-MAN 90:10 were not observed.

### B. In Vitro Aerosol Performance and Stability

Aerodynamic particle size distribution of TFF-VCZ-MAN powder formulations was determined by a NGI, and the FPF (% of metered) is presented in FIG. 11. Based on the FPF (% of metered dose) data, TFF-VCZ-MAN powder formulations consisting of 90 to 97% (w/w) voriconazole exhibited the highest aerosolization. FPF (% of metered dose) of TFF-VCZ-MAN 97:3 was significantly higher (p<0.05) than that of TFF-VCZ with 66% improvement in FPF (% of metered dose). Aerosol performance of TFF-VCZ-MAN powders containing 90 to 97% (w/w) voriconazole were not significantly different (p>0.05). Aerosol performance of TFF-VCZ-MAN powder formulations declined when greater than 10% (w/w) mannitol was included in the composition.

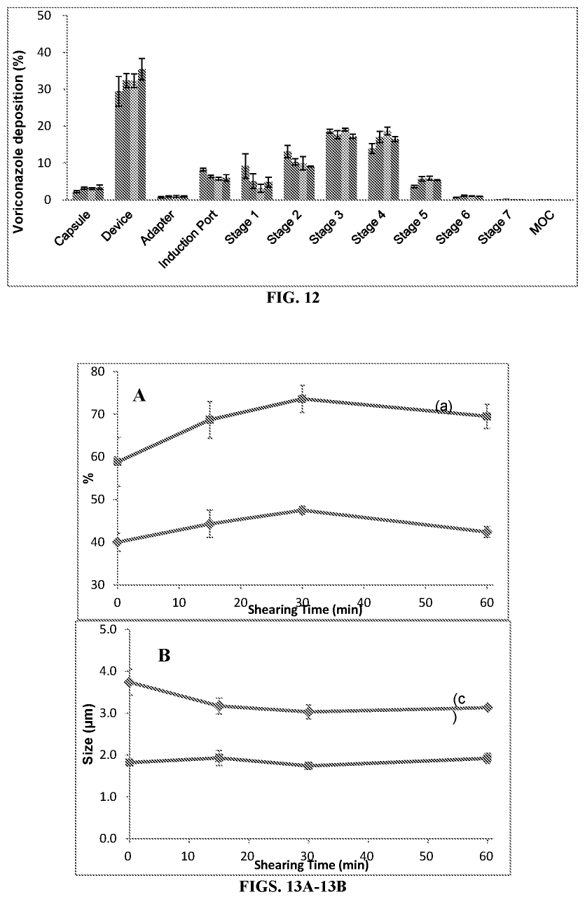

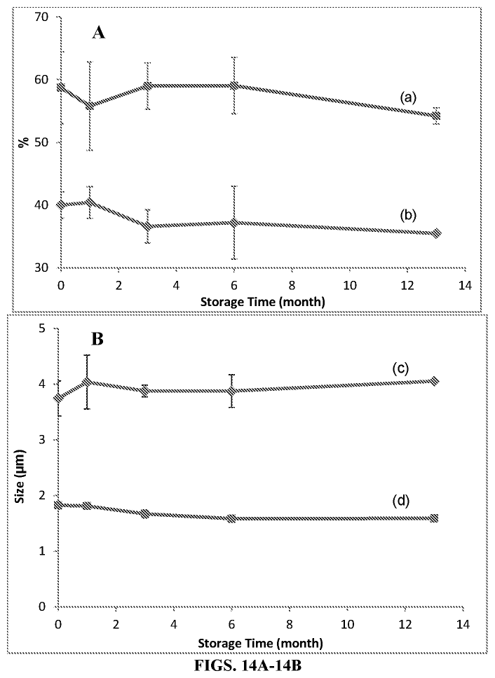

The influence of physical force on aerosol performance of TFF-VCZ-MAN 95:5 powder formulation was also investigated by measuring FPF using the NGI. As shown in FIGS. 12 and 13, particle size distribution and aerosol performance changes by different time of shear force was monitored. At 15, 30, and 60 min, FPFs (% of metered) were 44.3, 47.5, and 42.4% respectively, and FPFs (% of delivered dose) were 68.7, 73.6, and 69.5% respectively. The initial value before applying shear force was 40.0% for FPF (% of metered dose) and 58.8% for FPF (% of delivered dose). While a change in MMAD was also observed from 3.7 μm at the initial time to 3.2, 3.0, and 3.1 μm at 15, 30, and 60 min, respectively, no significant change was found for the GSD.

A stability study was performed at 25° C./60% RH, and the purity and aerosol performance changes of TFF-VCZ-MAN 95:5 powder formulation were monitored for 13 months as shown in FIG. 14. Purity of voriconazole in TFF-VCZ-MAN 95:5 was maintained, and no degradant was detected during test period of time. To compare aerosol performance over the stability study, FPF (% of metered), FPF (% of delivered), MMAD, and GSD were compared at each time point. There was no statistically significant difference on FPF (% of metered) for 13 months, as well as FPF (% of delivered) (both p>0.05). While GSD after 1 month decreased from the initial value (p<0.05), MMAD did not present any differences for 13 months (p>0.05).

### C. Dissolution of Voriconazole Dry Powder Formulations

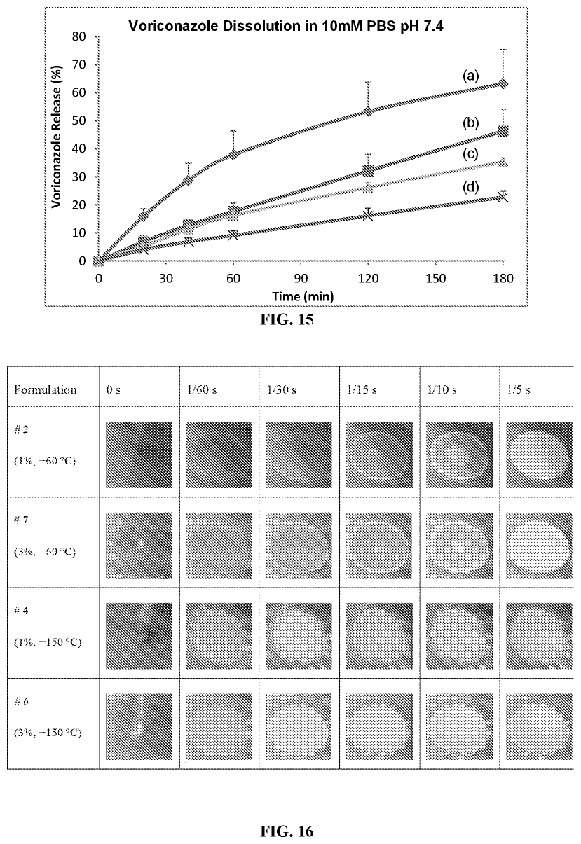

For dissolution testing of TFF-VCZ-MAN powder formulations, pH 7.4 PBS was used as the receptor media, and the top of donor chamber of the Franz-cells was covered with parafilm to prevent loss of dissolution media by evaporation. The dissolution rate of crystalline TFF-VCZ-MAN 95:5 was compared with amorphous TFF—VCZ-PVPK25 25:75, and the crystalline dry powder showed significantly slower cumulative drug release over the test time period (p<0.05) as shown in FIG. 15. Cumulative voriconazole release at 3 hours for amorphous TFF—VCZ-PVPK25 was 63.2%, while that for crystalline TFF-VCZ-MAN 95:5 was only 22.8%. Cumulative voriconazole released at 3 hours for TFF-VCZ-MAN 25:75 and TFF-VCZ-MAN 50:50 was 46.3 and 35.3%, respectively.

### D. Characterizations of Voriconazole Dry Powder Formulations

Voriconazole (Beinborn et al. 2012b; Ramos and Diogo 2016) and mannitol (Yu et al. 1998) have a high tendency of crystallization, and glass transition temperatures below room temperature. Therefore, TFF-VCZ-MAN was hypothesized to be crystalline unless there are strong intermolecular interactions between voriconazole and mannitol to prevent crystallization. The TFF-VCZ-MAN powder formulations were crystalline based on the XRPD data and the sharpness of 1D CP-MAS spectra, indicating that there are not sufficiently strong interactions between voriconazole and mannitol.

While XRPD is useful to characterize the crystallinity of powders, it may not be able to detect low amounts of amorphicity in the formulations. Therefore, mDSC was conducted on TFF-VCZ-MAN powders, and it was shown that TFF-VCZ-MAN dry powders were crystalline, since only two endothermic melting peaks of voriconazole and mannitol were detected. However, melting point depression was observed for mannitol especially in the TFF-VCZ-MAN 95:5. The low heat of fusion of mannitol in TFF-VCZ-MAN 95:5 could have occurred because of a relatively low amount of mannitol dissolved in melted voriconazole before a temperature reaches the melting point of mannitol. Also, mannitol particles in TFF-VCZ-MAN 95:5 are typically 100-200 nm, and these nanoscale particles can lower the heat of fusion. To confirm a potency of mannitol in TFF-VCZ-MAN powders that showed melting point depression, the molecular ratio between voriconazole and mannitol was determined by ¹H-NMR. While NMR is commonly used for qualitative analysis, quantitative NMR analysis is also applicable (Espina et al. 2009; Pauli et al. 2012). The experimental molecular ratios between voriconazole and mannitol matched well with the theoretical values in both of TFF-VCZ-MAN 95:5 and TFF-VCZ-MAN 50:50. Moreover, ¹³C and ¹⁹F ssNMR have often been used to confirm crystalline polymorphism and identify low levels of amorphous drug substance in solid dosage forms (Correa-Soto et al. 2017; Offerdahl et al. 2005). The identical peak positions and line widths of voriconazole resonances in ¹³C and ¹⁹F CP-MAS spectra of TFF-VCZ and TFF-VCZ-MAN 90:10 confirm the crystallinity and suggest no quantifiable amorphous content.

FTIR was used to study chemical interactions between voriconazole and mannitol. The hydroxyl group of voriconazole is related to its degradation pathway (Shaikh and Patil 2012), and it could be the most active site if there are any intermolecular interactions. If this occurred, this would shift the FT-IR peaks of voriconazole ranging between 3100 cm⁻¹ and 3500 cm⁻¹ (Silverstein et al. 2005). There are two peaks corresponding to voriconazole in this range, and they are at 3118.9 cm⁻¹ and 3198.4 cm⁻¹. These two peaks are observed in all of the TFF-VCZ-MAN and TFF-VCZ powder formulations, and no shift of these peaks was discovered. In case of mannitol in this range, a peak at 3276.6 cm⁻¹ was observed, and no shift was observed. If there are interactions between mannitol and aromatic secondary amines of voriconazole, peak shifts can be noticed between 1230 cm⁻¹ and 1300 cm⁻¹ (Silverstein et al. 2005). Four peaks from voriconazole at 1241.5 cm⁻¹, 1248.8 cm⁻¹, 1268.5 cm⁻¹, and 1277.6 cm⁻¹ were detected in this range, but no significant peak shift was found when mannitol was included in the voriconazole powder formulations. Therefore, these FT-IR data support that there is no or very weak interactions between voriconazole and mannitol in TFF-VCZ-MAN powder formulations.

While FT-IR is typically utilized to identify conformation and intermolecular interactions, ssNMR can provide more in-depth atomic-level information for structural investigation (Tian et al. 2017). In this research, 1D ¹³C and ¹⁹F CP-MAS were utilized to investigate conformational changes. All voriconazole peaks showed no difference in chemical shifts between TFF-VCZ and TFF-VCZ-MAN 90:10. Moreover, 2D ¹³C-¹H HETCOR spectra were acquired for investigating structural perturbations at a better resolution. This result confirms no ¹³C chemical shift change in the direct dimension. With the given resolution, chemical shifts of all aliphatic and aromatic protons in the indirect dimension also exhibit no observable changes. Besides, 2D ¹³C-¹H HETCOR has been utilized for detecting drug substance-excipient interactions. No inter-molecular cross peaks, i.e. interactions, have been observed between voriconazole and mannitol at the given spectral intensity.

Two different shapes of particles were observed in TFF-VCZ-MAN powder formulations, and it was initially thought that the micron-size particles were voriconazole, and the porous matrices were mannitol, based on the observed particle morphologies of TFF-VCZ and TFF-MAN. To confirm this, chemical compositions of these particles were confirmed by SEM/EDX. However, the locations that detected oxygen, fluorine, and nitrogen overlapped with each other during the initial SEM/EDX run, presenting particles that consisted of both voriconazole and mannitol. The cause was later identified. Since the measuring depth of EDX is micron scale, the detection beam passed through all of particle depth of TFF-VCZ-MAN 50:50 powders tested. To overcome this problem, the powder was dispersed widely on carbon tape on a specimen holder, and a spot analysis was performed to determine chemical compositions of two different morphologies of particles. By spot analysis, the micron-size particle was identified as voriconazole nanoaggregates based on the chemical compositions of oxygen, nitrogen, and fluorine, while the porous matrix was identified as mannitol, showing chemical composition of oxygen without nitrogen and fluorine. Therefore, it was concluded that crystalline mannitol was phase-separated from crystalline voriconazole during the TFF process.

While the AFM image in FIG. 5 shows that TFF-VCZ-MAN powders are nanoaggregates, the BET data is also supportive for the formation of voriconazole nanoaggregates. When SEM images present that TFF-VCZ particles are much greater than high porous matrix of TFF-MAN, the specific surface area of TFF-MAN is only about twice greater than that of TFF-VCZ. This can be because voriconazole particles are nanoaggregates having more specific surface area than visually seen on the SEM image.

### E. Level of Mannitol Affects Aerosol Performance and Dissolution Rate

The amount of mannitol in the TFF-VCZ-MAN powders affected their morphology. When low amount of mannitol was included, submicron mannitol particles were formed by prevention of particle growth as a result of high supercooling during the TFF process (Engstrom et al. 2008). These particles existed on the surface of voriconazole nanoaggregates, and modified their surface texture. These submicron mannitol particles were not taken out from the surface of voriconazole nanoaggregates during aerosolization. This could be due to that it was difficult to remove nano-size particles from the surface. While cohesive and adhesion forces are proportional to the diameter of particles, removal forces are proportional to the cube of the diameter for gravitational, vibrational, and centrifugal forces (Hinds 1999). Therefore, submicron mannitol particles were difficult to separate from voriconazole nanoaggregates, and the rough surface texture of voriconazole nanoaggregates was maintained during aerosolization, resulting in greater aerosolization. As the amount of mannitol in TFF-VCZ-MAN powders increased, large porous mannitol matrices were produced. These did not only exist on the surface of voriconazole nanoaggregate particles, but also were surrounding them. Multiple voriconazole nanoaggregates were assembled as the large porous mannitol matrix caused them to remain together. These aggregate structures remained during aerosolization. As a result, these large aggregated particles decreased aerosol performance of TFF-VCZ-MAN powder formulations that contained more than 10% (w/w) of mannitol.

Aerosol performance of formulations for DPI significantly relies on cohesive and adhesive forces of the particles. These forces include van der Waals, surface tension of adsorbed liquid films, and electrostatic forces (Hickey et al. 1994). All these are influenced by particle shape and size, surface roughness/texture, relative humidity, temperature, duration and velocity of particle contact (Hinds 1999; Beach et al. 2002; Tan et al. 2016; Price et al. 2002). Among these forces, van der Waals forces are the most important (Hinds 1999). Since van der Waals forces are attractive forces induced by dipoles between molecules, they decrease greatly when the distance between surfaces of particles reaches the separation distance (Hinds 1999). Therefore, rougher surfaces reduce van der Waals forces critically by keeping further average particle distances. Surface roughness affects not only van der Waals forces, but also surface tension, which is induced by surface moisture. A smooth surface of particles and high relative humidity lead to stronger surface tension. Electrostatic force, however, relies on the particle size. Particles bigger than 0.1 μm can generate electrostatic force (Hinds 1999). This attractive electrostatic force is stronger with larger particles, and is also related with relative humidity; low humidity retains the charges on the particles for longer time. Still, the electrostatic force is typically considered smaller than van der Waals and surface tension forces (Hinds 1999). Hence, surface roughness and texture of particles plays a significant role in aerosol performance of formulations for DPI.

The morphological changes of the powder formulations caused by different amounts of mannitol notably affected the aerosol performance of TFF-VCZ-MAN powder formulations. The aerosol performance was altered by the change of cohesive and adhesive forces of particles, and lowering these forces are related with the reduced contact areas between particles (Beach et al. 2002), in addition to further distance between particles (Hinds 1999). By including low quantities of submicron mannitol particles, the contact areas of TFF-VCZ-MAN nanoaggregates was significantly reduced, and the distance between voriconazole particles were further apart as shown illustrations in FIG. 4. Compared to the TFF-VCZ powder, TFF-VCZ-MAN 99:1 powder showed a significant improvement in FPF (% of metered dose) (p<0.05). This improvement by the addition of mannitol continued up to 3% (w/w) of mannitol was added in the formulation. An increase of about 5% in FPF (% of metered dose) was achieved by the addition of 1% (w/w) mannitol to formulations containing 97% to 100% (w/w) of voriconazole. In addition, TFF-VCZ-MAN 95:5 powders exhibited about 30% higher emitted dose compared to TFF-VCZ powders (68% vs. 36% respectively, data not shown). This enhanced emitted dose was accomplished as a result of reduced adhesion forces of particles to the device. Since TFF produces TFF-VCZ-MAN powders that contain very small amounts of moisture (less than 0.1% w/w, data not shown), and voriconazole and mannitol are not hygroscopic, the surface tension forces are expected to be relatively low on these particles. Storing powders in low humidity environment can generate electrostatic forces, but these forces are considered much smaller than van der Waals and surface tension forces (Hinds 1999). Accordingly, reducing contact areas of particles and furthering particle distance by modifying surface textures were primarily involved in lowering cohesive and adhesive forces of the TFF-VCZ-MAN powder formulations that led to the aerosol performance improvement. Young et al. similarly described the relationship between aerosol performance and separation energy between particles (Young et al. 2002) that corresponds well with our results.

Different amounts of mannitol in the TFF-VCZ-MAN powders not only affected aerosol performance, but also dissolution rate. TFF-VCZ-MAN powders containing higher amount of mannitol exhibited increased dissolution rates, and this could be explained by faster wetting of the powders by mannitol. For TFF-VCZ-MAN powders including high amount of mannitol, the surrounding mannitol particles, that were enclosing voriconazole, were wetted and dissolved very quickly. Therefore, voriconazole nanoaggregates were surrounded by the dissolution media in a short time, and the dissolution rate became faster. SEM picture of TFF-VCZ-MAN 25:75 powders presented that most mannitol particles dissolved in less than 5 min on the Franz-cells, while submicron mannitol particles were still observed on the surface of voriconazole nanoaggregates from TFF-VCZ-MAN 95:5 powders. This represented that voriconazole nanoaggregates did not get wet quickly when only a small amount of mannitol was included to the powder formulations.

### F. Benefits of TFF Process

High potency nanoaggregates of voriconazole powder formulations were made by TFF. While DPI formulations without carriers have been reported previously (Yazdi and Smyth 2016a; Yazdi and Smyth 2016b), carriers are commonly included in DPI formulations. However, carrier based DPI formulations are generally low drug potency. Also, many factors, such as particle size (Du et al. 2014), size distribution (Steckel and Muller 1997), and surface morphology (Du et al. 2014; Flament et al. 2004) of carrier particles, influence the powder aerosol performance during aerosolization, and such factors have negative effects on deposited dose uniformity (Du et al. 2017). By using TFF, the maximum aerosol performance of TFF-VCZ-MAN nanoaggregates was attained with as low as 3% (w/w) mannitol; therefore the potency of optimized TFF-VCZ-MAN powder formulation can be up to 97% (w/w). This high drug potency with a very low level of excipient requires less powder to be delivered, and the issues, such as low potency and deposited dose nonuniformity, generally caused by carriers can be eliminated.

High potency DPI formulations can be also made by other techniques, such as milling, for example. Even though the size of particles produced by milling and suitable for lung delivery is a few microns, such particles are considered as single discrete micron-size particles. As nanoaggregates, voriconazole DPI formulations made by TFF can have significantly higher total lung absorption efficiency and uniformity of dose distribution based on the study by (Longest and Hindle 2017). These voriconazole nanoaggregates are expected to allow for better epithelial coverage where fungal colonies are present. TFF was able to produce nanoaggregates, because rapid nucleation with a freezing rate of up to 10,000 K/sec allowed for a narrower particle size distribution and lower Ostwald ripening, producing a larger number of nuclei and preventing particle growth during the freezing process (Engstrom et al. 2008; Overhoff et al. 2009). The small size of unfrozen channels and the rapidly increased viscosity of unfrozen solution (Engstrom et al. 2008) made similar size of voriconazole nanoaggregates.

Surface modification of particles can be also accomplished by TFF. Begat et al. previously reported surface modification of particles using hydrophobic materials, such as lecithin, leucine, and magnesium stearate. While particles processed by dry mechanical fusion processes, like mechanofusion, presented improved aerosol performance with or without carriers by lowering surface free energy (Begat et al. 2005; Begat et al. 2009), this process was based on blending of drug substances with force controlling agents, like lecithin, leucine, and magnesium stearate. Mechanofusion process requires mechanical energy input to the formulation, and can cause chemical instability of the drug. In addition, surface modification by blending may be applicable only to discrete micron-size particles, not nanoaggregates due to possible deaggregation of aggregates by blending. Kawashima et al. also reported surface modification of particles by various methods, such as mechanical sheared mixing, freezing, or spray drying (Kawashima et al. 1998). With a hydrophilic additive, such as light anhydrous silicic acid (AEROSIL), the surface of hydrophobic particles converted to hydrophilic, and the surface modified particles presented improved inhalation behaviors in vitro. However, this method uses discrete micron-sized drug particles, and cannot be used for nanoaggregates. Therefore, these discrete micron-sized particles processed by other methods cannot attain the enhanced uptake and microdosimetry of those nanoaggregates described by Longest and Hindle 2017. By TFF, however, energy input was not needed to modify surfaces of particles. Surface modification of voriconazole nanoaggregates by phase-separated, submicron mannitol particles, which individually existed on the surface of drug nanoaggregates, was carried out due to rapid freezing rate that prevents particle growth.

High potency (up to 97% w/w) nanoaggregates of crystalline voriconazole powder formulations intended for dry powder inhalation were successfully developed using TFF technology. A low amount of mannitol, used as a single excipient, favorably enhanced the aerosol performance of voriconazole nanoaggregates by the phase-separated submicron crystalline mannitol acting as a surface texture-modifying agent. Voriconazole dry powder for inhalation made by TFF is a viable local treatment option for invasive pulmonary aspergillosis with high aerosolization efficiency and drug loading while offering the potential benefits associated with deposition of nanoaggregates in the airway.

### Example 2—Materials and General Methods

### A. Materials

The following materials were purchased: Voriconazole (Carbosynth, San Diego, Calif.); Kollidon® 25 (D-Basf, Ludwigshafen, Germany); acetonitrile (HPLC grade, Fisher Scientific, Pittsburgh, Pa.); trifluoroacetic acid (TFA) (HPLC grade, Fisher Scientific, Pittsburgh, Pa.); Tuffryn Membrane Filter (25 mm, 0.45 μm, Pall Corporation, Port Washington, N.Y.). Filtered water (Evoqua, Warrendale, Pa.) was used, and pyrogen free mannitol, Pearlitol® PF, was generously donated from Roquette America Inc. (Geneva, Ill.).

### B. Preparation of Powder for Dry Powder Inhalation Using TFF

Mannitol and voriconazole (30 to 100% w/w) powders were dissolved in a mixture of acetonitrile and water (50:50 v/v), and the solid content in the solution was kept as 1% w/v. Approximately 15 μL of each solution was dropped from a height of 10 cm onto a rotating cryogenically cooled (−60° C.) stainless steel drum. The frozen samples were collected in a stainless steel container filled with liquid nitrogen, and transferred into a −80° C. freezer until transferred to a lyophilizer. A VirTis Advantage Lyophilizer (VirTis Company Inc., Gardiner, N.Y.) was used to remove the solvent. The samples were kept at −40° C. for 21 hours, and the temperature was slowly increased to 25° C. over 21 hours, and then kept at 25° C. for another 21 hours to dry. The pressure was kept at 100 mTorr during the drying process.

### C. X-Ray Powder Diffraction (XRPD)

Crystallinity of the powder samples was determined by X-ray diffraction (MiniFlex 600, Rigaku Co., Tokyo, Japan) measuring from 5 to 35°2θ (0.02° step, 3°/min, 40 kV, 15 mA).

### D. Scanning Electron Microscopy (SEM)

SEM (Zeiss Supra 40V SEM, Carl Zeiss, Heidenheim an der Brenz, Germany) was used to identify the surface morphology of the samples. An aliquot of powder was placed onto carbon tape, and sputter coated with 60/40 Pd/Au for 20 min before capturing the images.

E. Modulated Differential Scanning Calorimetry (mDSC)

Thermal analysis of the powder samples was studied by differential scanning calorimetry model Q20 (TA Instruments, New Castle, Del.) equipped with a refrigerated cooling system (RCS40, TA Instruments, New Castle, Del.). Modulated DSC was performed with modulation period of 50 sec, modulated amplitude of 1° C., and average heating rate of 5° C./min. Tzero pan and Tzero hermetic lid manufactured by TA Instruments were used to hold samples during the test, and a hole was made on the lid with 20 G syringe needle before placing the pan in the sample holder.

### F. Scanning Electron Microscopy-Energy Dispersive X-Ray Spectroscopy (SEM/EDX)

SEM/EDX (Hitachi 55500 SEM/STEM, Hitachi America, Tarrytown, N.Y.) was used to identify elements of the powders produced by TFF.

### G. Atomic Force Microscopy (AFM)

Two different types of atomic force microscopy were used during this study. 3-dimensional (3D) surface topography images of particles generated by TFF were obtained by Asylum MFP-3D AFM (Oxford Instruments, Oxfordshire, United Kingdom), equipped with an aluminum coated MikroMasch HQ:NSC15 cantilever (NanoWorld AG, Neuchatel, Switzerland), which has a resonance frequency of 325 kHz, force constant of 40 N/m, and typical tip radius of 8 nm. Powders were affixed to an AFM disc with carbon tape, and compressed nitrogen gas was used to blow out particles which did not adhere to the carbon tape firmly. Topography was carried out with tapping mode at a scan rate of 1.00 Hz, set point of 1.08 V, and integral gain of 20.0. Feedback filter, drive amplitude and drive frequency were optimized for each sample, and all images were collected with 512×512 resolution. Gwyddion software (Necas and Klapetek 2012) (64 bit Windows version 2.50) was used to generate 3D topography images.

To obtain the image of nanoaggregates, Park XE-100 AFM (Park systems, Suwon, Korea) was used, equipped with an aluminum coated Nanosensors PPP-NCHR cantilever (NanoWorld AG, Neuchatel, Switzerland), which has resonance frequency of 330 kHz, force constant of 42 N/m, and tip radius of less than 7 nm. 380 μm single side polished P-type silicon wafer was coated with Tween® 20 (VWR, Radnor, Pa.) prior to load powder samples for AFM. Tween® 20 (1.5% w/v) was previously dissolved in HPLC grade methanol (Fisher Scientific, Pittsburgh, Pa.). The solution was dropped on to the silicon wafer using a transfer pipette, and solution was removed by compressed nitrogen gas. Powder was put into a DP4 insufflator (Penn-Century Inc., Wyndmoor, Pa.), and aerosolized on to the silicon wafer using a 3 mL syringe. After aerosolized powder was loaded on the silicon wafer, compressed nitrogen gas was used to remove powder solids that were not strongly adhered to the silicon wafer. Tapping mode was applied to collect images of 512×512 resolution with a scan rate of 0.30 Hz. Other values for AFM were optimized for each sample. The topography image was processed by Gwyddion software (Necas and Klapetek 2012) (64 bit Windows version 2.50).

### H. Aerodynamic Particle Size Distribution Analysis

Aerodynamic particle size was determined by a Next Generation Pharmaceutical Impactor (NGI) (MSP Co. Shoreview, Minn.), connected with High Capacity Pump (model HCPS, Copley Scientific, Nottingham, UK) and Critical Flow Controller (model TPK 2000, Copley Scientific, Nottingham, UK). A #3 HPMC capsule (VCaps plus, Capsugel, Morristown, N.J.), containing TFF powder (approximately 5 to 10 mg), was placed into high resistant RS01 dry powder inhaler (Plastiape, Osnago, Italy), and dispersed into the NGI through the USP induction port at the flow rate of 60 L/min for 4 seconds per each actuation. The pre-separator was not used for entire test. NGI collection plates were coated with 2% w/v polysorbate 20 in methanol and allowed to dry for 20 min before use. After aerosolization, the powder was extracted with the mixture of water and acetonitrile (50:50 v/v), and analyzed voriconazole contents by HPLC. Mass median aerodynamic diameter (MMAD), geometric standard deviation (GSD), and fine particle fraction (FPF) were calculated based on the dose deposited on device, induction port, stages 1 through 7, and micro-orifice collector (MOC) using Copley Inhaler Testing Data Analysis Software (CITAS) version 3.10 (Copley Scientific, Nottingham, UK).

### I. High-Performance Liquid Chromatography (HPLC)

A Dionex Ultimate 3000 HPLC system (Sunnyvale, Calif.) and Shimadzu DGU 14A degasser (Shimadzu, Kyoto, Japan) were used to measure the quantity of voriconazole contents. A Waters Xbridge C18 column (4.6×150 mm, 3.5 μm) (Milford, Mass.) was used. The method details are as follows: an isocratic method for aerodynamic properties using a mobile phase of 40/60 (v/v) water/acetonitrile containing 0.1% (v/v) TFA and a flow rate of 0.8 mL/min for 4 min; and a gradient method for chemical degradants during stability study. For the gradient method, acetonitrile containing 0.1% (v/v) TFA was gradually increased from 25 to 95% (v/v) for 14 min, mixed with water containing 0.1% (v/v) TFA, and a flow rate was 0.8 mL/min. For both methods, the samples were analyzed at a detection wavelength of 254 nm at 25° C. Linearity was performed between 50 ng/mL and 100 μg/mL with using an injection volume of 15 μL.

J. Solution Nuclear magnetic resonance (Solution NMR)

¹H NMR was performed to calculate the weight ratio between voriconazole and mannitol of the TFF-VCZ-MAN powders. All ¹HNMR spectra were recorded in dimethyl sulfoxide-_{d6} (DMSO-_{d6}) at 600 MHz on a VNMR 600 (Varian, Palo Alto, Calif.) spectrometer at 25° C. Chemical shifts were recorded relative to 2.47 ppm of DMSO-d6.

K. Solid-State Nuclear Magnetic Resonance (ssNMR)

ssNMR Experiments were carried out on a Bruker Avance III HD 400 MHz spectrometer (Bruker, Billerica, Mass.) at 25° C., with a magic angle spinning (MAS) frequency of 12 kHz. Bruker 4 mm triple resonance HFX probe was utilized in the double-resonance modes tuned to ¹H/¹³C or ¹H/¹⁹F frequencies. All samples were packed under ambient conditions in 4 mm ZrO₂ rotors (Wilmad-LabGlass, PA). One-dimensional (1D)¹³C and ¹⁹F cross-polarization (CP) MAS experiment was conducted with a linearly ramped power level of 80-100 kHz during a 2 ms contact period on the ¹H channel for enhancing CP efficiency. High power SPINAL64 proton decoupling was used at a field strength of 80 kHz. Same power parameters, contact time, MAS frequency were employed for 2-dimensional (2D)¹³C-¹H CP heteronuclear correlation (HETCOR) experiments. Adamantine was used as an external standard for calibrating ¹³C chemical shift, with the ethyl ¹³C peak referenced at 38.48 ppm.

### L. Fourier-Transform Infrared Spectroscopy (FT-IR)

Nicolet™ iS™ 50 FT-IR equipped with Smart OMNI-Sampler™ (ThermoFisher Scientific, Waltham, Mass.) was used to study intermolecular activity between voriconazole and mannitol of TFF-VCZ-MAN powders. The measurement was performed with the sample as a dry powder, and a spectral range of 4000 to 700 cm⁻¹ was recorded at aperture of 150, resolution of 4, and scan numbers of 32.

### M. Brunauer-Emmett-Teller (BET) Specific Surface Area (SSA) Analysis

Monosorb™ rapid surface area analyzer model MS-21 (Quantachrome Instruments, Boynton Beach, Fla.) was used to measure SSA of TFF-VCZ-MAN powders by single-point BET method. Samples were outgassed with nitrogen gas at 20 psi at ambient temperature for 24 hours to remove surface impurities. A mixture of nitrogen/helium (30:70 v/v) was used as the adsorbate gas.

### N. Shear Force Resistance Test

To test shear force resistance of TFF-VCZ-MAN 95:5 powders, the powders were placed into a stainless steel container (inner diameter 2⅞ inch, height 4¼ inch), and pre-sheared by rolling the container at 85 rpm. The powder sample was taken at 15, 30, and 60 min, and the aerodynamic property was compared with the initial condition.

### O. Dissolution Test

An in vitro dissolution method was used to quantite dissolution of voriconazole from powders processed by TFF technology. Franz cell apparatus was used to enable differentiation of voriconazole release from powders produced by TFF. A Next Generation Pharmaceutical Impactor (NGI) (MSP Co. Shoreview, Minn.), connected with High Capacity Pump (model HCPS, Copley Scientific, Nottingham, UK) and Critical Flow Controller (Model TPK 2000, Copley Scientific, Nottingham, UK) was used to load aerosolized powders on a Tuffryn membrane filter (25 mm, 0.45 μm, Pall Corporation, Port Washington, N.Y.). Five nozzles at stage 2 on the lid of NGI were blocked with lab tape, and only 1 nozzle was left opened. A Tuffryn membrane filter was placed and fixed with lab tape on the collection cup under the opened nozzle at stage 2. A #3 HPMC capsule (VCaps plus, Capsugel, Morristown, N.J.), containing TFF powder (approximately 5 to 10 mg), was placed into high resistant RS01 dry powder inhaler (Plastiape, Osnago, Italy), and dispersed into the NGI through the USP induction port at the flow rate of 60 L/min for 4 seconds per each actuation. A pre-separator was not used. After aerosolization, the powder-loaded (approximately 0.5 to 1 mg) membrane filter was carefully removed from the collection cup, and placed on top of a receptor chamber of Franz cell that was previously filed with degassed 10 mM phosphate buffered saline (PBS), pH 7.4 (5 mL). A donor chamber was placed on the membrane filter, and the membrane filter was fastened between receptor and donor chambers with a pinch clamp. Parafilm was used to cover the top of donor chamber. Dissolution test was conducted at sink conditions at 37° C. while magnetic bars were stirring in receptor chambers. Dissolution media (150 μL) was withdrawn at timed intervals of 0, 20, 40, 60, 120, and 180 min for HPLC analysis without dilution. Fresh dissolution media was replaced after each sampling.

P. Collection of Aerosolized Particles and Preparation of SEM Samples During dissolution

The Fast Screening Impactor (FSI) (Copley Scientific, Nottingham, UK) connected with High Capacity Pump (model HCPS, Copley Scientific, Nottingham, UK) and Critical Flow Controller (model TPK 2000, Copley Scientific, Nottingham, UK) was used to record SEM images of aerosolized TFF-VCZ-MAN powders before and during the dissolution test. A #3 HPMC capsule (VCaps plus, Capsugel, Morristown, N.J.), containing the TFF powder (approximately 5 to 10 mg), was placed into a high resistance RS01 dry powder inhaler device (Plastiape, Osnago, Italy), and dispersed into a glass fiber filter (MSP Co. Shoreview, Minn.) set in the FSI to collect particles of aerodynamic size 5 μm or less. Once the particles were collected on the filter, they were transferred on to carbon tape, previously attached on the SEM specimen, by tapping the carbon tape on the filter, and the SEM image was recorded.

To record SEM images during the dissolution test, a glass fiber filter loaded with powders from the FSI was cut round (25 mm diameter). The glass fiber filter was then placed between a donor chamber and a receptor chamber of Franz cell, previously filled with PBS, pH 7.4, at 37° C. The filter was left on the Franz cell for 5 min, and placed in to a −80° C. freezer for 1 hour. A VirTis Advantage Lyophilizer (VirTis Company Inc., Gardiner, N.Y.) was used to remove the solvent at 25° C. for 5 hours. Carbon tape, previously attached on SEM specimen, was tapped on the glass fiber filter to transfer the TFF-VCZ-MAN powders, and SEM image was recorded as described previously.

### Q. Stability Study

TFF-VCZ-MAN 95:5 dry powders were pre-sheared in a glass bottle, as described in shear force resistance test. Between 7.6 mg and 8.4 mg of the pre-sheared powder was filled into a size #3 HPMC capsule (Capsugel, Morristown, N.J.). 14 capsules filled with powders were transferred in a scintillation vial, and the vial was purged with nitrogen gas for 20 sec before closing with a cap. The vial was sealed in an aluminum foil (13×15 cm), previously purged with nitrogen gas inside for 30 sec, and the aluminum foils were kept at 25° C./60% RH. Purity and aerosol performance were performed at each time point of 1, 3, 6, and 13 months.

### R. Statistical Analysis

Aerodynamic performance and cumulative drug release were compared for statistical analysis by the student t-test. P-value<0.05 was considered as significantly different. JMP® 10.0.0 was used to compare the significance of the data.

### Example 3: Scaled Up Production of Voriconazole Composition and Inhaler Testing

1. Results

### A. Monitoring the Cooling Process at −60° C. and −150° C.

Table 3 shows the different formulations and processing conditions. FIG. 16 shows images of the freezing process at two different temperatures. The solutions containing voriconazole and mannitol (95:5 w/w) in water/ACN (50:50 v/v) were used at solid loading with 1% and 3% (w/v) (formulations #2, 4, 6, and 7 in Table 3). At −60° C., both solutions at different solid loadings showed that the freezing process was completed, and thermal equilibrium was reached in 200 ms or less. Nucleation was observed at the edge of the sample disc at around 1/30 ms, but the freezing progressed from the center of the disc to its edge at −60° C. In contrast, nucleation was initiated at 1/60 ms or less for solutions with 1% and 3% (w/v) solid loadings at −150° C., and cooling progressed homogeneously throughout the sample disc. However, thermal equilibrium was not reached by 200 ms.

TABLE 3
List of formulations and processing parameters

| | | | TFF | Ratio of |
|---|---|---|---|---|
| Formu- | Ratio of | Solid | processing | water:aceto- |
| lation | voriconazole:mannitol | load | temperature | nitrile |
| number | (w/w) | (% w/v) | (° C.) | (v/v) |
| 1 | 95:5 | 1 | −60 | 30:70 |
| 2 | 95:5 | 1 | −60 | 50:50 |
| 3 | 95:5 | 1 | −60 | 70:30 |
| 4 | 95:5 | 1 | −150 | 50:50 |
| 5 | 95:5 | 2 | −150 | 50:50 |
| 6 | 95:5 | 3 | −150 | 50:50 |
| 7 | 95:5 | 3 | −60 | 50:50 |

### B. Physical Properties of Voriconazole Nanoaggregates Made by TFF

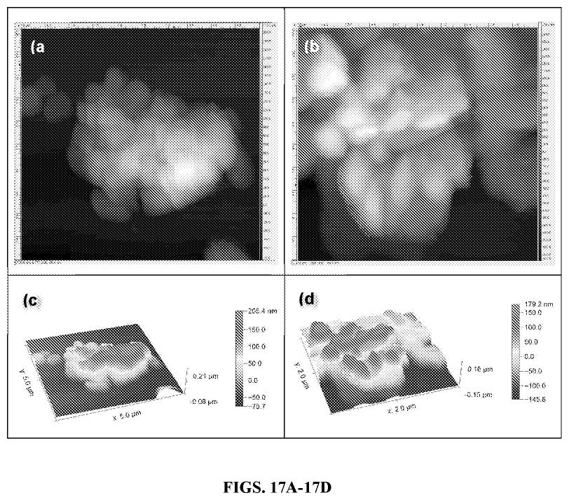

FIG. 17 presents high-resolution topography of voriconazole nanoaggregates processed at two different temperatures. It indicates that voriconazole nanoaggregates formed at a lower temperature (−150° C.) (formulation #4) consist of smaller nanoparticles. When processed at −150° C., nanoparticles as small as 200 nm were observed, while nanoparticles of around 500 nm were discovered at −60° C. (formulation #2).

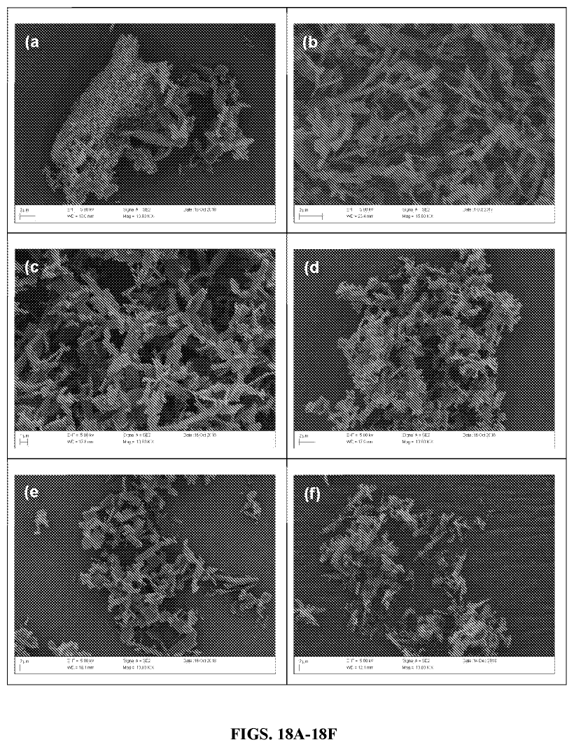

FIG. 18 compares the particle morphologies of voriconazole nanoaggregates formed using different processing parameters. When water/ACN (30:70 v/v) (formulation #1) was used as a solvent system, porous structured mannitol was observed with a particle size of over 20 μm. Voriconazole nanoaggregates produced with the other solvent systems showed surface texture modification of voriconazole particles by mannitol nanoparticles. Lower processing temperature resulted in smaller particles within the solid loading range tested (1˜3% w/v).

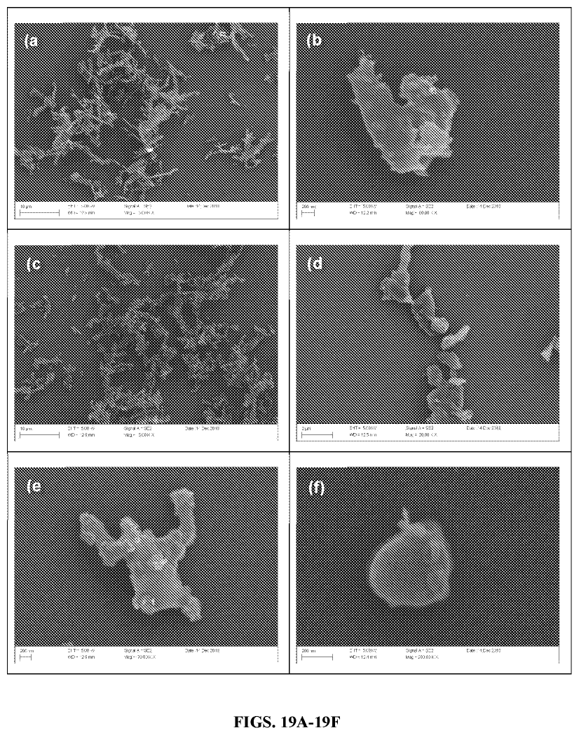

FIG. 19 shows SEM images of aerosolized voriconazole nanoaggregates made at −60° C. and −150° C. (formulations #7 and 6 respectively). It shows that the nanoaggregates consist of nanoparticles as small as 200 nm. While voriconazole nanoaggregates remained mainly as micro-sized nanoaggregates, irregularly shaped nanoaggregates that were not completely deaggregated after aerosolization were observed. The surface of these nanoparticles remained texture modified after aerosolization by the DP4 insufflator.

C. Comparison of Physical and Aerodynamic Properties with Scale-Up

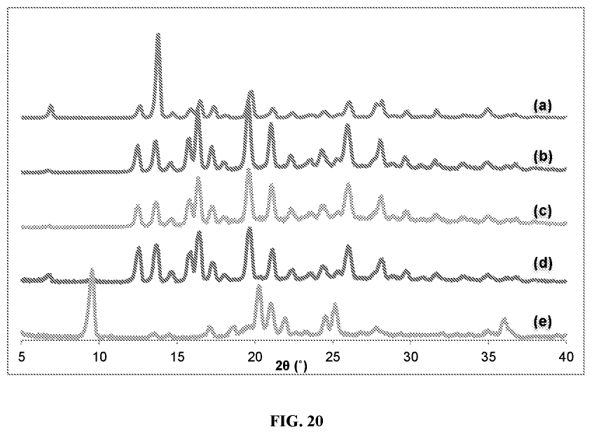

Table 4 shows the aerodynamic properties and moisture content of voriconazole nanoaggregates made in small (200 mg) and large scales (90 g). In addition, FIG. 20 compares the crystallinity of the various powder formulations. When the large scale is compared to the small scale, FPF (% of metered dose, 35.6% vs. 37.0%), FPF (% of delivered dose, 49.5% vs. 48.5%), and MMAD (3.69 μm vs. 3.52 μm) were not significantly different (p>0.05) when tested with a Plastiape® low resistance RS00 device at a flow rate of 60 L/min. Also, the moisture content of both batches was less than 0.1% (w/w) by TGA. XRPD spectra of voriconazole nanoaggregates did not show any pattern differences between the small and large scales.

TABLE 4
Comparison of physicochemical and
aerodynamic properties by scale

| Formulation | Voriconazole nanoaggregates formulation # 6 | |
|---|---|---|
| Test DPI | Plastiape low resistance RS-00 | |
| Flow rate (L/min) | 60 | |
| Batch scale | 90 g | 200 mg |
| MMAD (μm) | 3.69 ± 0.16 | 3.52 ± 0.06 |
| GSD (μm) | 1.82 ± 0.03 | N/A |
| FPF (% of metered dose) | 35.6 ± 1.9 | 37.0 ± 1.0 |
| FPF (% of delivered | 49.5 ± 2.3 | 48.5 ± 1.7 |
| dose) | | |
| SSA (m²/g) | 10.77 ± 0.62 | 8.65 ± 0.21 |
| Moisture contents | <0.1 | <0.1 |
| (% w/w) | | |

### D. In Vitro Aerosol Performance

i. By Cosolvent, Processing Temperature, and Solid Loading

NGI was used to evaluate effects of cosolvent, processing temperature, and solid loading on the aerosol properties of voriconazole nanoaggregates without conditioning. The results are presented in Table 5. A different ratio of water and acetonitrile in the cosolvent altered the aerosol properties of voriconazole nanoaggregates when the solid loading (1%) and processing temperature (−60° C.) were fixed (formulations #1-3). As the ratio of water was increased from 30% (v/v) to 50% (v/v), and 70% (v/v), the FPF (% of metered dose) was increased from 34.3% to 37.9%, and 45.6%. Also, FPF (% of delivered dose) was increased (53.1%, 61.2%, and 69.9% respectively), while the MMAD decreased (3.41, 3.31, and 3.09 μm respectively).

TABLE 5
Aerosol properties by solvent system, processing
temperature, and solid loading

| | FPF, | FPF, | | | |
|---|---|---|---|---|---|
| Formu- | metered | delivered | MMAD | GSD | ED |
| lation | (%) | (%) | (μm) | (μm) | (%) |
| 1 | 34.3 ± 1.7 | 53.1 ± 2.0 | 3.41 ± 0.25 | 1.80 ± 0.01 | 64.6 ± 2.4 |
| 2 | 37.9 ± 3.4 | 61.2 ± 5.0 | 3.31 ± 0.38 | 1.87 ± 0.05 | 62.2 ± 7.8 |
| 3 | 45.6 ± 2.9 | 69.9 ± 3.4 | 3.09 ± 0.17 | 1.76 ± 0.06 | 65.3 ± 3.6 |
| 4 | 46.7 ± 1.4 | 67.5 ± 2.0 | 3.27 ± 0.06 | N/A | 69.1 ± 2.0 |
| 5 | 41.3 ± 2.1 | 60.9 ± 5.3 | 3.24 ± 0.12 | N/A | 68.0 ± 2.6 |
| 6 | 37.0 ± 1.0 | 48.5 ± 1.7 | 3.52 ± 0.06 | N/A | 76.3 ± 0.8 |

Plastiape low-resistance RS-00 device at 60 L/min (n = 3; mean ± SD) Powder unconditioned

The influence of processing temperature was also confirmed. When the processing temperature was decreased from −60° C. to −150° C. while the solid loading (1%) and the cosolvent (water/ACN 50:50 v/v) were fixed (formulations #2 and 4), the FPF (% of metered dose) was significantly increased from 37.9% to 46.7% (p<0.05). However, the FPF (% of delivered dose) and MMAD did not change significantly (61.2% vs. 67.5% and 3.31 vs. 3.27 μm, respectively) (p>0.05).

Solid loading also impacts aerosol properties. As shown in Table 5, higher solid loading results in lower aerosol properties. As solid loading increases from 1% to 2%, and 3% (formulations #4-6), the FPF (% of metered dose) decreased from 46.7% to 41.3%, and 37.0% when the powder was not conditioned. The FPF (% of delivered dose) also decreased from 67.5% to 60.9%, and 48.5%. While the MMAD of 1% and 2% (formulations #4 and 5) were not significantly different (3.27 vs. 3.24 μm, p>0.05), 3% (formulation #6) resulted significantly larger MMAD (3.52 μm, p<0.05).

ii. By Device

The aerosol performance of voriconazole nanoaggregates (formulation #6) was evaluated using four different types of Plastiape devices: low and high resistance RS00, and low and high resistance RS01. Table 6 shows the assessment of the influence of different flow rates on aerosol performance. With a flow rate of 90, 60, and 30 L/min, the low resistance RS00 device showed a FPF (% of metered dose) of 48.6%, 45.8%, and 27.0% and an FPF (% of delivered dose) of 63.7%, 63.9%, and 48.9% respectively. MMAD was increased from 3.22 to 3.36 and 4.32 μm as the flow rate decreased from 90 to 60 and 30 L/min. The high resistance RS00 device showed an FPF (% of metered dose) of 34.7% at 60 L/min and 30.7% at 30 L/min. The MMAD of the high resistance RS00 device was 3.76 μm at 60 L/min and 3.83 μm at 30 L/min. The FPF (% of metered dose) of the low resistance RS01 device at a flow rate of 90, 60, and 30 L/min was 40.1%, 35.8%, and 27.0%, respectively, and the MMAD was 4.28, 4.37, and 5.34 μm, respectively. The high resistance RS01 showed an FPF (% of metered dose) of 31.7% at 60 L/min and 20.2% at 30 L/min, while MMAD was 4.48 and 5.06 μm respectively. In general, the low resistance device presented higher aerodynamic performance at the same flow rate, and the RS00 device resulted in better performance compared to the RS01 during the in situ aerosol performance test.

TABLE 6
Aerosol properties by devices

| | | Flow | FPF, | FPF, | | | |
|---|---|---|---|---|---|---|---|
| | | rate | Metered | Delivered | MMAD | GSD | ED |
| Device | Resistance | (L/min) | (%) | (%) | (μm) | (μm) | (%) |
| RS00 | Low | 90 | 48.6 ± 2.2 | 63.7 ± 2.0 | 3.22 ± 0.12 | N/A | 76.3 ± 2.0 |
| | | 60 | 45.8 ± 0.8 | 63.9 ± 0.8 | 3.36 ± 0.02 | N/A | 71.7 ± 1.4 |
| | | 30 | 27.0 ± 2.8 | 48.9 ± 1.0 | 4.32 ± 0.05 | N/A | 55.2 ± 5.8 |
| | High | 60 | 34.7 ± 2.3 | 55.0 ± 5.7 | 3.76 ± 0.20 | 1.69 ± 0.06 | 63.3 ± 3.8 |
| | | 30 | 30.7 ± 2.8 | 61.9 ± 3.4 | 3.83 ± 0.11 | 1.52 ± 0.02 | 49.5 ± 3.0 |
| RS01 | Low | 90 | 40.1 ± 2.0 | 49.0 ± 1.6 | 4.28 ± 0.12 | N/A | 81.7 ± 1.6 |
| | | 60 | 35.8 ± 2.6 | 49.5 ± 4.8 | 4.37 ± 0.30 | N/A | 72.5 ± 2.2 |
| | | 30 | 27.0 ± 3.6 | 35.3 ± 4.1 | 5.34 ± 0.28 | 1.87 ± 0.07 | 76.4 ± 2.2 |
| | High | 60 | 31.7 ± 0.7 | 48.5 ± 1.5 | 4.48 ± 0.10 | 1.65 ± 0.02 | 65.5 ± 0.9 |
| | | 30 | 20.2 ± 0.8 | 40.1 ± 2.8 | 5.06 ± 0.11 | 1.72 ± 0.02 | 50.6 ± 2.7 |

iii. By Dose

The effect of different dosage loadings on aerosol performance using high resistance RS00 and high resistance RS01 devices were tested. The results are presented in Table 7. When the loading dose increases from 10 mg to 15 mg, and 20 mg, the aerosol properties of FPF (% of metered dose) (34.7%, 33.8%, and 31.8%, respectively), FPF (% of delivered dose) (55.0%, 55.5%, and 51.5%, respectively), and MMAD (3.76, 3.77, and 3.84 μm, respectively) were not changed significantly (p>0.05) using the high resistance RS00 device at 60 L/min. However, the high resistance RS01 device at 60 L/min showed a significant difference (p<0.05) between a 10 mg and a 20 mg loading dose with an FPF (% of metered dose) (31.7% vs. 25.3%), FPF (% of delivered dose) (48.5% vs. 37.4%), and MMAD (4.48 vs. 5.21 μm).

TABLE 7
Aerosol properties by dose

| | Flow | Loading | FPF, | FPF, | | | |
|---|---|---|---|---|---|---|---|
| | rate | dose | metered | delivered | MMAD | GSD | ED |
| Device | (L/min) | (mg) | (%) | (%) | (μm) | (μm) | (%) |
| RS-00 | 60 | 10 | 34.7 ± 2.3 | 55.0 ± 5.7 | 3.76 ± 0.20 | 1.69 ± 0.06 | 63.3 ± 3.8 |
| High | | 15 | 33.8 ± 1.9 | 55.5 ± 2.9 | 3.77 ± 0.12 | 1.69 ± 0.02 | 60.9 ± 0.2 |
| resistance | | 20 | 31.8 ± 5.6 | 51.5 ± 9.6 | 3.84 ± 0.37 | 1.81 ± 0.15 | 61.6 ± 0.6 |
| RS-01 | 60 | 10 | 31.7 ± 0.7 | 48.5 ± 1.5 | 4.48 ± 0.10 | 1.65 ± 0.02 | 65.5 ± 0.9 |
| High | | 15 | 28.4 ± 2.2 | 43.2 ± 5.0 | 4.78 ± 0.35 | 1.72 ± 0.05 | 65.9 ± 2.5 |
| resistance | | 20 | 25.3 ± 2.5 | 37.4 ± 4.3 | 5.21 ± 0.31 | 1.76 ± 0.05 | 67.6 ± 2.3 |

TABLE 8
Aerosol properties by device based upon pressure drop

| | | Pressure | Flow | FPF, | FPF, | | % in |
|---|---|---|---|---|---|---|---|
| | | drop | rate | metered | delivered | MMAD | Device, |
| Device | Resistance | (kPa) | (L/min) | (%) | (%) | (μm) | metered |
| RS00 | High | 4 | 58 | 32.8 ± 1.7 | 49.1 ± 4.1 | 4.12 ± 0.22 | 33.1 ± 3.3 |
| | | 2 | 39 | 31.2 ± 4.5 | 51.5 ± 5.8 | 4.31 ± 0.19 | 39.6 ± 2.0 |
| | | 1 | 27 | 30.0 ± 2.2 | 55.0 ± 6.1 | 4.20 ± 0.31 | 45.3 ± 2.9 |
| RS01 | High | 4 | 68 | 29.8 ± 1.4 | 41.2 ± 3.2 | 4.86 ± 0.22 | 27.6 ± 2.6 |
| | | 2 | 45 | 27.4 ± 5.4 | 45.7 ± 9.5 | 4.70 ± 0.46 | 39.9 ± 0.6 |
| | | 1 | 32 | 24.7 ± 0.7 | 42.0 ± 0.1 | 4.97 ± 0.01 | 41.2 ± 1.6 |
| | Medium | 4 | 87 | 32.6 ± 2.9 | 41.4 ± 4.2 | 4.81 ± 0.30 | 21.2 ± 1.4 |
| | | 2 | 60 | 32.7 ± 2.1 | 44.2 ± 2.1 | 4.82 ± 0.13 | 25.9 ± 1.6 |
| | | 1 | 40 | 31.7 ± 3.3 | 46.3 ± 4.8 | 4.73 ± 0.27 | 31.6 ± 2.3 |
| | High | 4 | 68 | 31.6 ± 3.3 | 48.9 ± 6.3 | 4.45 ± 0.31 | 35.1 ± 2.2 |
| | (pinched | | | | | | |
| | by RS00) | | | | | | |

n = 3
Loading: 15.0 mg/capsule (fixed)

### 2. Discussion

### A. Processing Design Space of Voriconazole Nanoaggregates Made by TFF

Processing parameters within the design space of the freezing process used in TFF must be considered and their impact understood during development and subsequent scale-up, and includes: the solvent system, processing temperature, solid loading, and batch size. A low resistance RS00 device at a flow rate of 60 L/min was utilized to determine the processing design space since aerosolization by the low resistance RS00 device was more dependent on the inhalation flow rate and characteristics of the formulations. The dependency was able to distinguish aerosolization from formulations made by different processing design parameters.

### B. Solvent System

The physicochemical properties of amorphous solid dispersions of danazol made using TFF were not affected by the two different solvents (tert-butanol and acetonitrile (Overhoff et. al., 2007) that were used; however, the crystallinity, morphology, and aerosol performance of voriconazole with PVP K12 or PVP K30 produced using TFF were different, depending on the solvent compositions, which included water and 1,4-dioxane (Beinbom al., 2012). The cosolvent system of water and acetonitrile used during this research was adopted to develop tacrolimus formulations and voriconazole formulations made using TFF (Watts et al., 2013; Moon et al., 2019).

While a difference in crystallinity was not observed, a disparity in morphology was found in different solvent compositions. In addition, a significant trend in aerodynamic properties was observed when solvent composition changed. Without powder conditioning, the solvent composition containing higher portion of water showed enhanced aerosolization. This result may relate to two factors: viscosity and cryo-phase separation of the cosolvent system.

In water and the acetonitrile cosolvent system, viscosity increases with a larger portion of water (Thompson et al., 2006; Cunningham et al., 1967). During the freezing process, high viscosity can impede the movement of molecules. Therefore, molecules are distributed more homogeneously in the frozen state, and solute concentration in the unfrozen channels may not increase significantly. Low viscosity of the solvent permits more movement of molecules during the freezing process, and molecular agglomeration may occur. As a result, solute concentration in the unfrozen channels increases. Since voriconazole powders made by TFF are crystalline nanoaggregates, higher solute concentration can induce the production of larger nanoparticles due to the shorter distance between molecules.

Even though TFF involves ultra-rapid supercooling, the freezing process of the water/ACN solvent system requires up to 200 ms at −60° C. Thus, during this freezing time of 200 ms, there is a chance of a higher degree of molecular agglomeration with a low-viscosity solvent that results in lower aerosol performance. This trend was also observed in the previous study by Beinborn et al. about voriconazole made using TFF (Beinborn al., 2012). When crystalline voriconazole powders containing PVP K12 or PVP K30 were produced with water and a 1,4-dioxane binary solvent system, higher aerosolization was obtained with TFF particles made with 1,4-dioxane/water (20:80 v/v) compared to particles made with 1,4-dioxane/water (50:50 v/v). Although the viscosity of 1,4-dioxane is higher than that of water, the viscosity of 1,4-dioxane/water (20:80 v/v) is higher than 1,4-dioxane/water (50:50 v/v) (Besbes et al., 2009). Therefore, the viscosity of the cosolvent system is one of the factors that influence aerosol performance after lyophilization.

The prevention of cryo-phase separation is the second possibility of enhanced aerosol performance by means of a cosolvent system with a higher portion of water. The cosolvent system consists of water and acetonitrile, and it is well known for its phase separation during the freezing process when 35-88% (v/v) of acetonitrile is included (Gu et al., 1994; Zarzycki et al., 2006). Once the phase separation occurs below −1.32° C. (Zarzycki et al., 2006), unfrozen solvent is separated into an 88% (v/v) acetonitrile phase and a 65% (v/v) water phase, and solutes can move to the phase in which the solutes have higher solubility (Gu et al., 1994).

This cryo-phase separation occurred in formulation #1, which was processed with water/ACN (30:70 v/v) at −60° C. The 5% (w/w) mannitol in the voriconazole nanoaggregates acts as a surface texture-modifying agent (Moon et al., 2019). Therefore, the mannitol is observed on the surface of crystalline voriconazole nanoaggregates as nanoparticles, as shown in SEM images of the other formulations in FIG. 18. However, around 20 μm, porous mannitol particles were observed, which have the same morphology as TFF-mannitol (Moon et al., 2019). With mannitol particles of this size generated in formulation #1, the effect of surface texture-modification by mannitol is diminished, because less amount of mannitol is available to act for surface texture-modification, thereby causing poor aerosolization.

Even though TFF's supercooling can minimize phase separation and generate very small ice channels (Moon et al., 2016) due to ultra-rapid freezing, the processing temperature of −60° C. allows for low-level phase separation of the water/ACN (30:70 v/v), which prompts agglomeration and an increased concentration of mannitol during the freezing processing time (up to 200 ms). However, water/ACN (70:30 v/v) does not phase separate during freezing (Gu et al., 1994; Zarzycki et al., 2006). Therefore, the agglomeration and concentration increase induced by cryo-phase separation is unlikely.

### C. Processing Temperature

In addition to solvent compositions, the processing temperature also influences the aerosol performance of crystalline voriconazole nanoaggregates made using TFF. Lower processing temperature leads to a higher degree of supercooling, thus generating smaller ice channels and preventing particle growth (Overhoff et al., 2009; Engstrom et al., 2008). A temperature of −150° C. in this research showed much faster nucleation with ultra-rapid supercooling. This supercooling at −150° C. generated smaller nanoparticles in the voriconazole nanoaggregates, consisting of nanoparticles as small as 200 nm, observed using both AFM and SEM by prevention of particle growth. In contrast, when processed at −60° C., a particle size of around 500 nm was observed using AFM. When voriconazole nanoaggregates consist of smaller nanoparticles, they are more likely to deaggregate into smaller particles during inhalation, leading to enhanced aerosol performance.

Interestingly, the enhanced level of aerosol performance induced by higher supercooling at −150° C. is equivalent to the higher performance induced by the cosolvent system of water/ACN (70:30 v/v) at lower supercooling at −60° C. FPF (% of metered dose) and MMAD are not significantly different (p>0.05) under these two processing conditions (formulations #3 and #4).

### D. Solid Loading

Increasing the solid loading is one of the ways to reduce processing time in the manufacture of powder formulations using TFF. However, higher solid loading typically impairs aerosol performance. The bulk density of voriconazole nanoaggregates produced with 1% (w/v) solid loading is about 30 mg/cm³ with no conditioning or physical shearing. Therefore, in an attempt to accelerate the manufacturing process, the solid loading was increased to 3% (w/v), which corresponds to a bulk density of 30 mg/cm³. Two optimized processing parameters were initially applied when solid loading was increased to 3% (w/v): a water/ACN (70/30 v/v) solvent system and a processing temperature of −150° C. However, due to the low solubility of voriconazole in water/ACN (70/30 v/v), a solid loading of 3% (w/v) was not applicable. Therefore, water/ACN (50/50 v/v) was chosen.

While a bulk density with 3% (w/v) solid loading induced a similar bulk density from 1% (w/v) after lyophilization, the result was lower aerosol performance before powder conditioning. The performance, however, was enhanced with proper conditioning comparable to voriconazole nanoaggregates made at 1% (w/v) solid loading with optimized processing parameters (formulation #4). Accordingly, aerosolization tests were conducted with formulation #6 with powder conditioning.

### E. Batch Size

Until recently, the TFF process was applied using either a syringe or a separation funnel to feed solutions dropwise. The result is that solutions required more time to freeze. This was a major hindrance to the scale-up of the TFF process. To expedite the freezing process, a 2-channel peristaltic pump was applied to produce amorphous meloxicam using the TFF process (Jermain et al., 2019). During this research on voriconazole, however, the number of channels increased to eight, and the feeding rate of the solutions was optimized at 25 mL/min. Simultaneously, the cryogenic drum rotating rate was increased from 10 rpm to 20 rpm to avoid frozen sample discs overlapping each other at the higher feeding rate.

The increase in the rotation rate shortened the time in which frozen samples remained on the cryogenic drum. This time decreased from 4 s to 2 s before they were collected in a tray containing liquid nitrogen. However, due to the ultra-rapid freezing of TFF, the freezing process typically requires less than a few hundred milliseconds, (Overhoff et. al., 2007) and we did not expect the increased cryogenic drum rotation rate to influence the freezing process. For formulation #6 in this research, nucleation occurred in less than 1/60 s (in FIG. 18), and thermal equilibrium of the frozen sample was reached in less than 2 s before they were collected in the tray.

The ultra-rapid supercooling at −150° C. accelerates the nucleation rate and increases the number of ice crystals formed (Rambhatla et al., 2004; Overhoff et al., 2009). Thus, homogeneous nucleation was observed throughout the frozen sample. Moreover, since the size of the droplets is similar, regardless of scale, the freezing process is independent from different scales, and the outcomes of the frozen samples do not differ significantly. With a similar freezing process overall, the physicochemical and aerodynamic properties on a small scale were comparable to larger scales.

After deciding to use a peristaltic pump in the TFF process scale-up, the lyophilizer capacity was also tested. The data in Table 4 also confirm that the lyophilization of 90 g of the voriconazole nanoaggregates by the 3-shelf AdVantage Pro lyophilizer did not differ from the 200 mg of voriconazole nanoaggregates lyophilized by the 1-shelf AdVantage 2.0 lyophilizer. Therefore, a scale-up of the TFF process using a peristaltic pump at a feeding solution flow rate of 25 mL/min is suitable when using a 3-shelf AdVantage Pro lyophilizer.

F. Interaction of Devices with Voriconazole Nanoaggregates by Different Devices and Flow Rates

During the development of pharmaceutical products delivered by DPI, device design or selection is as important as formulation development in terms of aerosol performance. The same powder formulation can aerosolize differently with different DPI devices (Parumasivam et al., 2017). In this research, commercially available Plastiape RS01 and RS00 devices, which are applied to many DPI products in the market or in development, were tested (Armer et al., 2016; Elkins et al., 2014; Roscigno et al., 2017). RS01 and RS00 devices adopt the same delivery technology: A capsule is lifted from its housing and spins at high speed (Dry Powder Inhaler RS01: How to Use: Plastiape; [Available from: plastiape.com/en/content/1635/dry-powder-inhaler-rs01-how-use). However, powders in the RS01 device evacuate the capsule through two holes, while the RS00 device discharges powders through eight smaller holes of the capsule with longer mouthpiece. In comparison, the overall aerosol properties of voriconazole nanoaggregates using the RS00 device are superior when compared using the same flow rate with the same type of resistance.

This higher performance achieved using the RS00 device could be due to the smaller holes created by the piercing system of the RS00 device. When voriconazole nanoaggregates leave the capsule, the smaller holes may assist the deaggregation of large voriconazole nanoaggregates, and their smaller size results in a smaller MMAD and a higher FPF. This could be a unique feature of voriconazole nanoaggregates for DPI because they are composed of brittle nanoaggregates. Other powder formulations for DPI made using spray drying or milling may not be considered nanoaggregates. Therefore, the size of the holes when the powders evacuate the capsule may not significantly affect overall performance.

Comparing the low and high resistance of the RS01 and RS00 devices, both low resistance devices generally performed better than the high resistance devices at the flow rates of 60 and 30 L/min. Also, low resistance devices presented higher ED relative to high resistance devices. However, powder deaggregation and microdispersion with a low-resistance device relies on the patient's inhalation flow rate, (Dal Negro RW, 2015) causing variations in aerosolization over different inhalation flow rates. This was also observed in both low resistance devices in this research. Although a flow rate of 90 L/min achieved the maximum aerosolization with low resistance RS00 device, a significant decrease (18.8%) of FPF (% of metered dose) was observed at 30 L/min. A similar trend when using the low resistance RS01 device was observed. The decrease in FPFs (% of metered dose) from a flow rate of 60 to 30 L/min was also significant (8.8%) when using low resistance RS01. However, the FPF (% of metered dose) using the high resistance RS00 device differed by only 4.0% between flow rates of 60 and 30 L/min, and the MMADs were not significant (p>0.05), although a notable difference in ED was observed. The high resistance RS00 device showed inhalation flow rate independence between 60 and 30 L/min that is caused by a sufficient regimen of turbulence (Dal Negro RW, 2015). Therefore, even though the low resistance RS00 device performs better from an aerosol properties standpoint, these properties may vary significantly among individual patients, thus inducing efficiency variations. In the case of the high resistance RS01 device, however, flow rate independence between 60 and 30 L/min was not observed, confirming that the smaller holes in the RS00 device contribute to the aerosolization of voriconazole nanoaggregates.

### G. By Different Dosage Loading

The bulk density of voriconazole nanoaggregates prior to conditioning is typically around 30 mg/cm³ regardless of solid loading (1 to 3% w/v) of the solutions before freezing using TFF. However, the bulk density increases gradually up to 100 mg/cm³ with conditioning or externally applied physical shear stress. The voriconazole nanoaggregates were conditioned to have a bulk density around 60 mg/cm³, and the influence of powder fill level were evaluated with a size #3 HPMC capsule. Since a capacity volume of a #3 capsule is 0.3 mL, the maximum amount of voriconazole nanoaggregates that can be inserted in a capsule is approximately 20 mg after conditioning. Therefore, the aerosol performance of voriconazole nanoaggregates was evaluated with a dose range of 10-20 mg per capsule. The high resistance RS00 and RS01 devices were utilized at a flow rate of 60 L/min, and the Tukey-Kramer HSD test was performed to compare the results between different powder levels.

While the high resistance RS00 device did not show a significant difference (p>0.05) in FPF (% of metered dose), FPF (% of delivered dose), and MMAD, the performance between 10 mg and 20 mg using the high resistance RS01 device was different (p<0.05). The consistency of aerosol performance using the high resistance RS00 device may be the result of smaller holes that can help aerosolize particles in a narrow distribution in the case of voriconazole nanoaggregates.

### 3. Materials and Methods

### A. Materials

Voriconazole USP was purchased from Aurobino Pharma Ltd. (Hyderabad, India). HPLC grade of acetonitrile (ACN), methanol, and trifluoroacetic acid (TFA) were purchased from Fisher Scientific (Pittsburgh, Pa.). In-house filtered water (Evoqua, Warrendale, Pa.) was used, and pyrogen-free mannitol, Pearlitol® PF, was donated from Roquette America Inc. (Geneva, Ill.).

### B. Preparation of Powder Formulations

Voriconazole (95% w/w) and mannitol (5% w/w) were dissolved in a mixture of acetonitrile and water (30:70, 50:50, or 70:30 v/v) with solid content in the solution of 1-3% (w/v). The solution was sonicated until a clear solution was obtained. The solution was then dropped from a height of approximately 10 cm onto a rotating cryogenically cooled (−60° C. or −150° C.) stainless steel drum. For the small scale, a 10 mL syringe with a syringe needle (18 gauge) was used to feed the solution onto the drum. For the large-scale process, a Masterflex® L/S® peristaltic pump (Cole-Parmer, Vernon Hills, Ill.) equipped with Masterflex® L/S® High-performance Precision Platinum-Cured Silicon pump tubing (size 16, Cole-Parmer, Vernon Hills, Ill.) was used to deliver solution onto the drum at a flow rate of 25 mL/min. During the freezing process, the frozen samples were collected in a stainless steel lyophilizer tray filled with liquid nitrogen and transferred to a −80° C. freezer to remove excess liquid nitrogen before transferring the sample to a lyophilizer. A VirTis Advantage 2.0 or VirTis Advantage Pro shelf lyophilizer (VirTis Company Inc., Gardiner, N.Y.) was used to sublime the solvents and dry the samples. During the primary drying process, the shelves were kept at −40° C. for 20 h, and the temperature of the shelves was linearly increased to 25° C. over 20 h, then kept at 25° C. for 20 h. The secondary drying was performed at 25° C. for 20 h. The pressure was kept at 100 mTorr during the lyophilization process.

### C. X-Ray Powder Diffraction (XRPD)

Powder crystallinity was identified using X-ray diffraction (MiniFlex 600, Rigaku Co., Tokyo, Japan) measuring from 5-40°2θ (0.02° step, 2°/min, 40 kV, 15 mA).

### D. Scanning Electron Microscopy (SEM)

SEM (Zeiss Supra 40V SEM, Carl Zeiss, Heidenheim an der Brenz, Germany) was used to determine the surface morphology of the powder samples and to identify nanoparticles after aerosolization. For identification of the surface morphology, an aliquot of powder was placed onto carbon tape and sputter coated with 60/40 Pd/Au with a thickness of 20 nm before capturing images. For the determination of nanoparticles, 1-2 mg of powder was placed into a DP4 insufflator (Penn-Century Inc., Wyndmoor, Pa.) and aerosolized onto the 380 μm single-side polished P-type silicon wafer using a 3 mL syringe and sputter coated with 60/40 Pd/Au with a thickness of 5 nm before capturing images.

### E. Atomic Force Microscopy (AFM)

To obtain the images of nanoaggregates, Asylum MFP-3D AFM (Oxford Instruments, Oxfordshire, United Kingdom) was utilized, which was equipped with a gold-coated MikroMasch Hi'Res-C15/Cr—Au cantilever (nanoWorld AG, Neuchâtel, Switzerland), which has a resonance frequency of 325 kHz, a force constant of 40 N/m, and a typical tip radius of 1 nm. A DP4 insufflator (Penn-Century Inc., Wyndmoor, Pa.) was utilized to affix powders onto the silicon wafer. 1-2 mg of powder were placed into the insufflator, and the powder aerosolized it onto the 380 μm single-side polished P-type silicon wafer using a 3 mL syringe. After the powder was loaded, the excess powder that did not strongly adhere to the wafer was blown out by compressed nitrogen gas. Topography was carried out with a tapping mode at a scan rate of 1.00 Hz. Other values for AFM were optimized for each sample. The images were collected using a 512×512 resolution and processed by Gwyddion software (64-bit Windows version 2.50) (Necas and Klapetek, 2012).

### F. Powder Conditioning

1.3 g of powder was added into a 60 mL Pyrex bottle. The bottle was rolled at 60 rpm for 30 min to shear the powders and was stored in a desiccant at room temperature.

### G. Aerodynamic Particle Size Distribution Analysis

Aerodynamic properties of the powder were measured by a Next Generation Pharmaceutical Impactor (NGI) (MSP Corporation, Shoreview, Minn.) equipped with a Critical Flow Controller (model TPK, MSP Corporation. Shoreview, Minn.) and a High Capacity Pump (model HCPS, MSP Corporation, Shoreview, Minn.). Approximately 5-20 mg of the powder formulation was inserted into a #3 HPMC capsule (Vcaps® plus, Capsugel®, Morristown, N.J.) and dispersed by either a Plastiape RS01 or an RS00 DPI into the NGI through the USP induction port with a total 4 L volume of airflow. The pre-separator was not employed. 1.5% (w/v) polysorbate 20 in methanol was applied to the NGI collection plates to coat and dry them for 20 min before use. After dispersal, the powder was extracted using the mixture of water and acetonitrile (50:50 v/v) containing 0.1% (v/v) TFA to analyze the voriconazole contents using HPLC. The MMAD, the geometric standard deviation (GSD), and the fine particle fraction (FPF) were calculated using Copley Inhaler Testing Data Analysis Software (CITDAS) version 3.10 (Copley Scientific, Nottingham, UK).

### H. High-Performance Liquid Chromatography (HPLC)

For quantitative analysis of the voriconazole contents, a Dionex Ultimate 3000 HPLC system (Sunnyvale, Calif.) was used connected to a Shimadzu DGU 14A degasser (Shimadzu, Kyoto, Japan) and a Waters Xbridge C18 column (4.6×150 mm, 3.5 μm) (Milford, Mass.). An isocratic method with a mobile phase of 40/60 (v/v) water/acetonitrile containing 0.1% (v/v) TFA at a flow rate of 0.8 mL/min for 4 min at 25° C. was used. The sample concentration was ascertained using a wavelength of 254 nm. A linearity study of the standard curve between voriconazole concentrations of 62.5 ng/mL and 500 μg/mL was conducted with an injection volume of 7 μL.

### I. Brunauer-Emmett-Teller (BET) Specific Surface Area (SSA) Analysis

To measure SSA, a Monosorb™ rapid surface area analyzer model MS-21 (Quantachrome Instruments, Boynton Beach, Fla.) was utilized. The powder formulation samples were outgassed using nitrogen gas at 20 psi at ambient temperature for over 24 h. A mixture of nitrogen and helium (30:70 v/v) was used as the adsorbate gas.

### J. Thermal Gravimetric Analysis (TGA)

TGA was performed to measure moisture content of the powder formulations. A Mettler Thermogravimetric Analyzer, Model TGA/DSC 1 (Columbus, Ohio) was used. Approximately 2-5 mg of the sample was placed in an alumina crucible (Mettler-Toledo, Columbus, Ohio) and covered with a crucible lid. The crucible was heated from 25° C. to 150° C. at a ramp rate of 5° C./min. The moisture content of the sample was calculated by comparing the decrease in the sample weight between 25° C. and 125° C.

### K. Photographs of Freezing Under Different Temperatures

To monitor freezing rate differences between different processing temperatures by TFF, the freezing process was captured by Canon DSLR camera, model EOS Rebel SL1 (Canon USA, Melville, N.Y.) equipped with 18-55 mm IS STM lens (Canon USA, Melville, N.Y.) at a frame rate of 60 frames per second, with resolution of 1280×720. The captured images were cropped to approximately 200×200 to present only the samples.

### L. Statistical Analysis

A student t-test was applied to determine whether the aerodynamic properties were statistically different. A p-value<0.05 was considered as significantly different. JMP® 10.0.0 was applied to calculate the p-value of the data.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Patent Application No. 2010/0221343
Armer et al., WO2016057712A1, 2016.
Arora et al., Mol Pharm.; 12(6):2001-9, 2015.
Arora et al., Expert opinion on drug delivery; 13(2):183-93, 2016.
Beach et al., J Colloid Interface Sci.; 247(1):84-99, 2002.
Begat et al., KONA Powder Part J.; 23:109-21, 2005.
Begat et al., J Pharm Sci.; 98(8):2770-83, 2009.
Beinborn al., Int J Pharmaceut.; 429(1-2): 46-57, 2012.
Beinborn et al., European journal of pharmaceutics and biopharmaceutics: Official Journal of Arbeitsgemeinschaft fur Pharmazeutische Verfahrenstechnik e V.; 81(3):600-8, 2012a.
Beinborn et al., Int J Pharmaceut.; 429(1-2): 46-57, 2012b.
Besbes et al., J Mol Liq.; 145:1-4, 2009.
Borro et al., Transplant Proc.; 40(9):3090-3, 2008.
Carvalho et al., European journal of pharmaceutics and biopharmaceutics: Official Journal of Arbeitsgemeinschaft fur Pharmazeutische Verfahrenstechnik e V.; 88(1):136-47, 2014.
Chiou and Riegelman, J Pharm Sci.; 60(9):1281-302, 1971.
Correa-Soto et al., Journal of pharmaceutical and biomedical analysis.; 146:86-95, 2017.
Cunningham et al., J. Chem Eng Data.;12(3):336-7, 1967.
Dal Negro R W, Multidiscip Respir Med.;10(1):13, 2015.
DiNunzio et al., Mol Pharm.;5(6):968-80, 2008.
Dry Powder Inhaler RS01: How to Use: Plastiape; [Available from: plastiape.com/en/content/1635/dry-powder-inhaler-rs01-how-use.
Du et al., Evaluation of Granulated Lactose as a Carrier for DPI Formulations: Effect of Drug Loading.: 743-6, 2014.
Du et al., J Pharm Sci.; 106(1):366-76, 2017.
Elkins et al., Open Respir Med J.; 8:1-7, 2014.
Engstrom et al., Pharm Res.; 25(6):1334-46, 2008.
Engstrom et al., Pharm Res.; 25(6):1334-46, 2008.
Espina et al., Chemical research in toxicology; 22(2):299-310, 2009.
Flament et al., Int J Pharmaceut.; 275(1-2): 201-9, 2004.
Gu et al., Sep Technol.; 4:258-61, 1994.
Hickey et al., Pharm Tech.; 18:58-64, 1994.
Hilberg et al., Eur Respir J; 40(1):271-3, 2012.
Hinds W C., Aerosol technology: properties, behavior, and measurement of airborne particles. 2nd ed. New York: Wiley; xx, 483 p. p., 1999.
Johnston et al., USA patent PCT/US2008/067766. 2015 Mar. 3, 2015.
Kawashima et al., Pharm Res.; 15(11):1748-52, 1998.
Lang et al., Mol Pharm.;11(1):186-96, 2014.
Lang et al., J Drug Deliv Sci Tec.;24(2):205-11, 2014.
Le and Schiller, Curr Fungal Infect Rep.; 4(2):96-102, 2010.
Liu et al., European journal of pharmaceutics and biopharmaceutics: official journal of Arbeitsgemeinschaft fur Pharmazeutische Verfahrenstechnik e V.; 96:132-42, 2015.
Longest and Hindle, Pharm Res.; 34(10):2049-65, 2017.
Moon et al, Mol Pharm. 2019.
Moon et al., Respir Drug Deliv.; 3:611-6, 2016.
Necas and Klapetek, Dent Eur J Phys.;10(1):181-8, 2012.
Necas and Klapetek, Dent Eur J Phys.;10(1):181-8, 2012.
O'Donnell et al., Drug Dev Ind Pharm.;39(2):205-17, 2013.
Offerdahl et al., J Pharm Sci.; 94(12):2591-605, 2005.
Overhoff et al., Pharm Res.; 25(4167-75, 2008.
Overhoff et al., European journal of pharmaceutics and biopharmaceutics: official journal of Arbeitsgemeinschaft fur Pharmazeutische Verfahrenstechnik e V.; 65(1):57-67, 2007b.
Overhoff et al., Int J Pharmaceut.;336(1):122-32, 2007a.
Overhoff et al., J Drug Del Sci Tech.; 19(2):89-98, 2009.
Overhoff et al., J Drug Del Sci Tech.;19(2):89-98, 2009.
Overhoff et. al., European journal of pharmaceutics and biopharmaceutics: official journal of Arbeitsgemeinschaft fur Pharmazeutische Verfahrenstechnik e V.; 65(1):57-67, 2007.
Parumasivam et al., Aaps J; 19(1):191-202, 2017.
Pauli et al., J Nat Prod.; 75(4):834-51 2012.
Price et al., Int. J Pharmaceut.; 246(1-2): 47-59, 2002.
Purvis et al., Aaps Pharmscitech.; 8(3): E58, 2007.
Rambhatla et al., Aaps Pharmscitech., 5(4): e58, 2004.
Ramos and Diogo, Int J Pharmaceut.; 501(1-2): 39-48, 2016.
Roscigno et al., WO2017192993A1, 2017.
Shaikh and Patil, Sci Pharm.; 80(4):879-88 2012.
Silverstein et al., Spectrometric identification of organic compounds. 7th ed. Hoboken, N.J.: John Wiley & Sons; x, 502 p. p., 2005.
Sinha et al., Nanomedicine; 9(1):94-104, 2013.
Sinswat et al., European journal of pharmaceutics and biopharmaceutics: official journal of Arbeitsgemeinschaft fur Pharmazeutische Verfahrenstechnik e V.; 69(3):1057-66, 2008.
Steckel and Muller, Int J Pharm.;154(1):31-7, 1997.
Tan et al., Pharm Res.; 33(8):1923-35, 2016.
Thakkar et al., Hum Vaccin Immunother.;13(4):936-46, 2017.
Thompson et al., J Chromatogr A.; 1134(1-2): 201-9, 2006.
Tian et al., The journal of physical chemistry B.; 121(25):6108-16, 2017.
Tolman et al., Eur J Pharm Biopharm.; 72(1):199-205, 2009a.
Tolman et al., Antimicrobial agents and chemotherapy; 53(6):2613-5, 2009b.
Wang et al., Aaps Pharmscitech.;15(4):981-93, 2014.
Watts et al., Int J Pharmaceut.; 384(1-2): 46-52, 2010.
Watts et al., Pharm Res.;30(3):813-25, 2013.
Watts, et al., 2013
Wisniewski R., Spray Drying Technology Review. 45th International Conference on Environmental Systems; 12-16 Jul. 2015; Bellevue, Wash. 2015.
Wong Y L, Ann Acad Med Singapore; 22(1):99-102, 1993.
Yang et al., Int J Pharmaceut.; 361(1-2): 177-88, 2008.
Yazdi and Smyth, Int J Pharm.; 511(1):403-14, 2016a.
Yazdi and Smyth, Int J Pharm.; 502(1-2): 170-80, 2016b.
Young et al., J Pharm Pharmacol.; 54(10):1339-44, 2002.
Yu et al., J Pharm Sci.; 87(6):774-7, 1998.
Zarzycki et al., The international journal of the Japan Society for Analytical Chemistry; 22(3):453-6, 2006.

Zhang et al., European journal of pharmaceutics and biopharmaceutics: official journal of Arbeitsgemeinschaft fur Pharmazeutische Verfahrenstechnik e V.; 82(3):534-44, 2012.

What is claimed is:

1. A pharmaceutical composition comprising:
(A) a therapeutic agent; and
(B) an excipient, wherein the excipient comprises less than about 10% by weight of the pharmaceutical composition;
wherein the pharmaceutical composition is formulated as a nanoaggregate comprising nanoparticles of the therapeutic agent and the surface of the nanoparticles of the therapeutic agent contains discrete domains of the excipient and wherein the discrete domains of the excipient reduce the contact area between the nanoparticles of the therapeutic agent.

2. The pharmaceutical composition of claim 1, wherein the therapeutic agent is present in a crystalline form.

3. The pharmaceutical composition of claim 1, wherein the therapeutic agent is present in an amorphous form.

4. The pharmaceutical composition according to any one of claims 1-3, wherein the excipient comprises from about 9% w/w to about 1 w/w of the pharmaceutical composition.

5. The pharmaceutical composition of claim 4, wherein excipient comprises from about 6% w/w to about 2% w/w of the pharmaceutical composition.

6. The pharmaceutical composition of claim 5, wherein the excipient comprises about 3% w/w of the pharmaceutical composition.

7. The pharmaceutical composition of claim 5, wherein the excipient comprises about 5% w/w of the pharmaceutical composition.

8. The pharmaceutical composition according to any one of claims 1-7, wherein the discrete domains of the excipient comprise one or more non-continuous domains of the excipient on the surface.

9. The pharmaceutical composition according to any one of claims 1-7, wherein the discrete domains of the excipient comprise a contiguous and continuous layer of the excipient.

10. The pharmaceutical composition according to any one of claims 1-9, wherein the excipient is water-soluble.

11. The pharmaceutical composition according to any one of claims 1-10, wherein the excipient is a sugar alcohol.

12. The pharmaceutical composition of claim 11, wherein the excipient is mannitol.

13. The pharmaceutical composition according to any one of claims 1-12, wherein the excipient is present as a nano-domain in the pharmaceutical composition.

14. The pharmaceutical composition of claim 13, wherein the nano-domain of the excipient have a size from about 50 nm to about 500 nm.

15. The pharmaceutical composition of claim 14, wherein the size of the excipient nano-domain is from about 100 nm to about 200 nm.

16. The pharmaceutical composition according to any one of claims 1-15, wherein the pharmaceutical composition has a mass median aerodynamic diameter from about 1.5 to about 7.5 μm.

17. The pharmaceutical composition of claim 16, wherein the mass median aerodynamic diameter is from about 2.5 to about 6.5 μm.

18. The pharmaceutical composition according to any one of claims 1-17, wherein the pharmaceutical composition does not include a wax excipient.

19. The pharmaceutical composition of claim 18, wherein the pharmaceutical composition does not include a hydrophobic excipient.

20. The pharmaceutical composition according to any one of claims 1-19, wherein the therapeutic agent is selected from the group comprising anticancer agents, antifungal agents, psychiatric agents such as analgesics, consciousness level-altering agents such as anesthetic agents or hypnotics, nonsteroidal anti-inflammatory drugs (NSAIDS), anthelminthics, beta agonists, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives.

21. The pharmaceutical composition of claim 20, wherein the therapeutic agent is an antifungal agent.

22. The pharmaceutical composition of claim 21, wherein the antifungal agent is an azole antifungal drug.

23. The pharmaceutical composition of claim 22, wherein the azole antifungal drug is voriconazole.

24. The pharmaceutical composition according to any one of claims 1-23, wherein the pharmaceutical composition further comprises one or more additional excipients.

25. The pharmaceutical composition according to any one of claims 1-24, wherein the pharmaceutical composition further comprises one or more additional therapeutic agents.

26. The pharmaceutical composition according to any one of claims 1-25, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transdermally, vaginally, in crèmes, in lipid compositions, via a catheter, via a lavage, via continuous infusion, via infusion, via inhalation, via injection, via local delivery, or via localized perfusion.

27. The pharmaceutical composition of claim 26, wherein the pharmaceutical composition is formulated for administration via inhalation.

28. The pharmaceutical composition according to any one of claims 1-27, wherein the pharmaceutical composition is formulated for use with an inhaler.

29. The pharmaceutical composition of claim 28, wherein the inhaler is a fixed dose combination inhaler, a single dose dry powder inhaler, a multi-dose dry powder inhaler, multi-unit dose dry powder inhaler, a metered dose inhaler, or a pressurized metered dose inhaler.

30. The pharmaceutical composition of claim 29, wherein the inhaler is a capsule-based inhaler.

31. The pharmaceutical composition according to any one of claims 28-30, wherein the inhaler is a low resistance inhaler.

32. The pharmaceutical composition according to any one of claims 28-30, wherein the inhaler is a high resistance inhaler.

33. The pharmaceutical composition according to any one of claims 28-32, wherein the inhaler is used with a flow rate from about 10 L/min to about 150 L/min.

34. The pharmaceutical composition of claim 33, wherein the flow rate is from about 20 L/min to about 100 L/min.

35. The pharmaceutical composition according to any one of claims 28-34, wherein the inhaler has a pressure differential is from 0.5 kPa to about 5 kPa.

36. The pharmaceutical composition of claim 35, wherein the pressure differential is 1 kPa, 2 kPa, or 4 kPa.

37. The pharmaceutical composition according to any one of claims 28-36, wherein the inhaler has a loaded dose from about 0.1 mg to about 50 mg.

38. The pharmaceutical composition of claim 37, wherein the inhaler has a loaded dose from about 0.1 mg to about 10 mg.

39. The pharmaceutical composition of claim 37, wherein the inhaler has a loaded dose from about 5 mg to about 50 mg.

40. The pharmaceutical composition of claim 39, wherein the loaded dose is from about 5 mg to about 25 mg.

41. The pharmaceutical composition according to any one of claims 1-40, wherein inhaler is configured to deliver one or a series of doses from one or more unit doses loaded sequentially.

42. The pharmaceutical composition of claim 41, wherein the inhaler is configured to deliver one dose from one unit dose.

43. The pharmaceutical composition of claim 41, wherein the inhaler is configured to deliver a series of doses from one unit dose.

44. The pharmaceutical composition of claim 41, wherein the inhaler is configured to deliver one dose each from a series of capsules loaded sequentially.

45. The pharmaceutical composition of claim 41, wherein the inhaler is configured to deliver a series of doses from a series of capsules loaded sequentially.

46. A method of treating or preventing a disease or disorder in a patient in need thereof comprising administering to the patient a therapeutically effective amount of a pharmaceutical composition according to any one of claims 1-45 comprising a therapeutic agent effective to treat the disease or disorder.

47. The method of claim 46, wherein the disease or disorder is in the lungs.

48. The method of either claim 46 or claim 47, wherein the disease or disorder is an infection.

49. The method according to any one of claims 46-48, wherein the infection is of a fungus.

50. The method of claim 49, wherein the therapeutic agent is an anti-fungal agent.

51. The method of claim 50, wherein the therapeutic agent is an azole anti-fungal agent.

52. The method of claim 51, wherein the therapeutic agent is voriconazole.

53. A method of preparing a pharmaceutical composition comprising:
(A) admixing a therapeutic agent and an excipient wherein the excipient is present in an amount of less than 10% w/w with a solvent to form a precursor solution;
(B) depositing the precursor solution onto a surface at a temperature suitable to cause the solvent to freeze; and
(C) removing the solvent to obtain a pharmaceutical composition.

54. The method of claim 53, wherein the solvent is a mixture of two or more solvents.

55. The method of claim 54, wherein the mixture of solvents comprises water.

56. The method of claim 55, wherein the solvent is an organic solvent.

57. The method of claim 56, wherein the organic solvent is acetonitrile.

58. The method of claim 56, wherein the organic solvent is 1,4-dioxane.

59. The method according to any one of claims 53-57, wherein the solvent is a mixture of water and an organic solvent.

60. The method of claim 59, wherein the solvent is a mixture of water and acetonitrile.

61. The method according to any one of claims 53-60, wherein the mixture of two or more solvents comprises from about 10% v/v to about 90% v/v of the organic solvent.

62. The method of claim 61, wherein the mixture comprises from about 40% v/v to about 60% v/v of the organic solvent.

63. The method of claim 62, wherein the mixture comprises about 50% v/v of the organic solvent.

64. The method of claim 61, wherein the mixture comprises from about 20% v/v to about 40% v/v of the organic solvent.

65. The method of claim 64, wherein the mixture comprises about 30% v/v of the organic solvent.

66. The method according to any one of claims 53-65, wherein the therapeutic agent and excipient comprises less than 10% w/v of the precursor solution.

67. The method of claim 66, wherein the therapeutic agent and excipient comprises from about 0.5% to about 5% w/v of the precursor solution.

68. The method of claim 67, wherein the therapeutic agent and excipient comprises about 1% w/v of the precursor solution.

69. The method of claim 67, wherein the therapeutic agent and excipient comprises about 3% w/v of the precursor solution.

70. The method according to any one of claims 53-69, wherein the surface is rotating.

71. The method according to any one of claim 53-70, wherein the temperature is from about 0° C. to about −200° C.

72. The method of claim 71, wherein the temperature is from about 0° C. to about −120° C.

73. The method of claim 72, wherein the temperature is from about −50° C. to about −90° C.

74. The method of claim 73, wherein the temperature is about −60° C.

75. The method of claim 72, wherein the temperature is from about −125° C. to about −175° C.

76. The method of claim 73, wherein the temperature is about −150° C.

77. The method according to any one of claims 53-76, wherein the solvent is removed at reduced pressure.

78. The method of claim 77, wherein the solvent is removed via lyophilization.

79. The method of claim 78, wherein the lyophilization is carried out at a lyophilization temperature from about −20° C. to about −100° C.

80. The method of claim 79, wherein the lyophilization temperature is about −40° C.

81. The method according to any one of claims 77-80, wherein the reduced pressure is less than 250 mTorr.

82. The method of claim 81, wherein the reduced pressure is about 100 mTorr.

83. The method according to any one of claims 53-82, wherein the method further comprises heating the pharmaceutical composition at reduced pressure.

84. The method of claim 83, wherein the pharmaceutical composition is heated to a temperature from about 0° C. to about 30° C.

85. The method of claim 84, wherein the temperature is about room temperature or about 25° C.

86. The method according to any one of claims 83-85, wherein the reduced pressure is less than 250 mTorr.

87. The method of claim 86, wherein the reduced pressure is about 100 mTorr.

88. The method according to any one of claims 83-87, wherein the reduced pressure is the same as the reduced pressure during the lyophilization.

89. A pharmaceutical composition prepared according to the methods of any one of claims 53-88.
